(19) United States
(12) Patent Application Publication
(10) Pub. No.: US 2008/0176962 A1
(43) Pub. Date: Jul. 24, 2008

(54) Methods and compositions for identifying cellular genes exploited by viral pathogens

(76) Inventors: Stanley N. Cohen, Stanford, CA (US); Daniel Rock, Stanford, CA (US); Annie Chang, Stanford, CA (US); Yanan Feng, Stanford, CA (US); Laszlo Zsak, Stanford, CA (US); Maria Elisa Piccone, Old Say Brook, CT (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/890,314

(22) Filed: Aug. 2, 2007

Related U.S. Application Data
(60) Provisional application No. 60/836,987, filed on Aug. 9, 2006.

(51) Int. Cl. A61K 47/00; C40B 30/06; C40B 40/02; A01K 67/027; A61P 43/00

(52) U.S. Cl. 514/789; 506/10; 506/14; 800/13

(57) ABSTRACT

Methods and compositions for rapidly identifying CGEPs required for viral infection of mammalian cells are provided. Also provided are methods of inhibiting viral infection of mammalian cells by inhibiting the activity of one or more CGEPs (e.g., as identified in accordance with methods of the invention) in the cells. Aspects of the invention further include specifically identified CGEPs implicated in mammalian cell infection of specific viruses, e.g., African Swine Fever Virus and Foot and Mouth Virus, and methods of modulating their activity to achieve viral resistance.

### CROSS-REFERENCE TO RELATED APPLICATIONS

Pursuant to 35 U.S.C. §119(e), this application claims priority to the filing date of the U.S. Provisional Patent Application Ser. No. 60/836,987 filed Aug. 9, 2006; the disclosures of which application is herein incorporated by reference.

### GOVERNMENT RIGHTS

This invention was made with Government support under contract N66001-01-1-8947 awarded by the Defense Advanced Research Projects Agency. The United States Government has certain rights in this invention.

### INTRODUCTION

Infection and propagation of viral pathogens requires not only the expression of genes carried by the pathogen, but also the cooperation of host genes, which may be collectively referred to as “cellular genes exploited by pathogens (CGEPs). Information encoded by the host genome is required for the virus to accomplish: 1) virus binding, 2) viral genome internalization and transport, 3) viral gene product expression, 4) viral protein processing, 5) viral genome replication, 6) viral pathogenic effects, and 7) virus morphogenesis and release.

Interfering with the dynamic genetic interactions between pathogen and host offers the potential to inhibit infection and prevent viral propagation and transmission. As such, there is intense interest in the identification of CGEPs, and specifically in methods of identifying CGEPs required for viral infection in mammalian cells.

### SUMMARY

Methods and compositions for rapidly identifying CGEPs required for viral infection of mammalian cells are provided. Also provided are methods of inhibiting viral infection of mammalian cells by inhibiting the activity of one or more CGEPs (e.g., as identified in accordance with methods of the invention) in the cells. Aspects of the invention further include specifically identified CGEPs implicated in mammalian cell infection of specific viruses, e.g., African Swine Fever Virus (ASFV) and Foot and Mouth Disease Virus (FMDV), and methods of modulating their activity, e.g., to achieve viral resistance.

### BRIEF DESCRIPTION OF THE FIGURES

The file of this patent contains at least one drawing executed in color. Copies of this patent with color drawings will be provided by the Patent and Trademark Office upon request and payment of the necessary fee.

FIG. 1. Extracellular ASFV virus titers of the reconstituted EST clones. The patterned bars represent the extracellular virus titer assayed in culture media used to grow reconstituted EST-expressing clones at the indicated hpi at an MOI of 10. The virus titer is expressed as the log 10 of the 50% tissue culture infectious dose (TCID50). The first two bars are controls and showed the same pattern when infected with ASFV: HeLatTA, a parental cell line, and an EST library that represents HeLatTA cells containing the collection of EST inserts in the parental cell line. Bars three through nine represent the HeLa cell clones, each of which contained a different EST insert and showed a reduced titer when infected with ASFV virus: BAT3S, C1qTNF6, MKPX, MYOHD1, an EST cell clone of unknown gene function, TOM40, and PPPIR12C.

FIG. 2. Virus titers in reconstituted EST clones taken at 72 hpi. The ASFV virus titer of reconstituted clones was grown in the absence and presence of doxycycline, and the sample was taken at 72 hpi. The names of the EST clones are shown below the bar. The y axis denotes the virus titer expressed as the log 10 of the 50% tissue culture infective dose (TCID50).

FIG. 3. Structural similarity between BAT3 domains and domains of other proteins, as determined by NCBI conservative domain homology analysis. The numbers on top denote the amino acid residues of the BAT3 peptide. The location of the BAT3 dominant negative peptide (BATdn) is shown as a box at the top of the BAT3 full-length protein. UBQ represents the ubiquitin-like peptide on BAT3; the region on the BAT3 peptide that is responsible for apoptotic activity contains a DEAD box, indicated as apoptosis. CAP and BAG1 indicate regions of homology to the adenylyl cyclase protein domain and BAG1 peptide, respectively. The box labeled ppro denotes the polyproline regions of BAT3, and the nuclear localization signal is shown in the box labeled N.

FIG. 4. Dot blot hybridization of genomic DNA isolated from ASFV-infected EST-expressing clones with ASFV DNA, as a measure of ASFV replication. Genomic DNA was isolated from BAT3 sense and antisense clones. One hundred nanograms of total DNA was diluted serially at 1:2, and 100-μl samples of the increasing dilutions were transferred to wells from the left to right orientation (columns 1 to 6). One hundred nanograms of DNA was spotted in wells marked 1, half of that amount was spotted in wells numbered 2, etc. Malawai (Mal) DNA is the ASFV viral DNA that served as a positive control; genomic DNA from a parental HeLa cell line was used as a negative control.

FIG. 5. cDNA sequence corresponding to BAT3 EST DNA and to the ORF of the fused BAT3 peptide. (A) The sequence of the BAT3 EST in the sense clone is bracketed by a pair of universal primers: the AEK reverse primer in the lined box is located at the 5′ side of the BAT3 sense sequence, and the AEK forward primer in the dotted box is positioned at the 3′ side of the BAT3 EST. The translational initiation codon of the BAT3 dominant peptide is underlined. (B) Putative ORF of the BAT3 peptide fused to a vector-encoded peptide corresponding to a segment of a woodchuck hepatitis virus DNA polymerase. The dotted box encompassing 71 amino acids is derived from the BAT3S EST, and the dashed box is the sequence of woodchuck hepatitis virus DNA polymerase encoded by the pLenti vector. The box formed by solid lines shows the NheI site where the EST was introduced into the vector, and the underlined sequence is the universal primer AEK forward, which is located on the 3′ side of the BAT3 EST sequence. The numbering at the left and right sides of the figure corresponds to amino acid and nucleotide sequence numbers of the ORF. The putative peptide contains 298 amino acids.

FIG. 6. Expression of BAT3 transcripts as measured by reverse transcription-PCR in HeLatTA, BAT3 antisense, and BAT3 sense cells. Three sets of primers derived from different exons were used to quantify BAT3 mRNA. The oligonucleotide sequence for each primer is shown in Materials and Methods. The y axis represents the percentage of expression relative to the GUS β-glucuronidase positive control.

FIG. 7. Viability of BAT3 EST-expressing cell lines in the presence of staurosporine. Approximately 10⁴ cells were grown for 24 h in each 96-well dish in different concentrations of staurosporine. The cells were then assayed for viability by the MTT method. Each point is the mean of three measurements, and the mean of the standard deviation for each point is shown as a bar in the figure. Three independent experiments were performed for this assay. The x axis shows the concentration of staurosporine used in the assay, and the y axis shows the percentage of cells that survived compared to cells without staurosporine treatment.

FIGS. 8A to 8E provide Tables 1 to 5 referenced in the Experimental Section, infra.

FIG. 9 Plaque titration assay on tTA LF-BK and Entpd6 cell clones. Cells were infected with FMDV O/UK/2001 at MOI 0.001 or 0.0001 (panel A) or different picornavirus (panel B) for 1 h at 37° C. and cultured under gum tragacanth. Cells were fixed and stained at 48 hpi. In panel C, FMDV resistance is induced in the Entpd6 cells in response to Dox. Cells were cultured in the absence (−) or presence (+) of 2 μg/ml Dox and viral resistance was examined by plaque assay.

FIG. 10. Overexpression of NTDPase 6 in E-clone 30 cells.

Wild type LF-BK-tTA and Clone 30 cells were transduced with a construct overexpressing NTDPase 6. Panel A) Western blot indicating expression of NTDPase 6 in untransduced wildtype cells (lanes 1 and 2) and in cells transduced with NTDPase 6 overexpression construct (lanes 3 and 4). Panel B) Plaque titration assay on tTA LF-BK (top panels) and Clone 30 (bottom panels) cells. Cells were infected with serial dilutions of FMDV O/UK/2001 alone (left panels) or with FMDV O/UK/2001 and NTDPase 6 overexpression construct (right panels) and cultured under gum tragacanth. Cells were fixed and stained at 48 hpi.

FIG. 11. FMDV growth curve in tTA LF-BK and Entpd6 cell clones. Cells were infected with FMDV O/UK/2001 at an MOI 10 (panel A) or MOI 0.1 (panel B) for 1 h at 37° C., as described under Materials and Methods, infra. Supernatants fluids were removed and titrated at the times indicated. Each data is the mean value for at least three independent experiments.

FIG. 12. FMDV RNA synthesis in infected tTA LF-BK and Entpd6 cell clones: Total RNA was extracted at different times post infection and subjected to northern blot analysis using ³²P-labelled antisense RNA derived from the 3D genomic region. Panel A) infection of E-clone 8 and clone 30 cells at MOI 10; panel B) infection E-clone 8 and clone 30 cells at MOI 0.1 in the absence of Dox and panel C) infection E-clone 30 cells at MOI 0.1 in the absence or presence of 2 μg/ml Dox. The lowers panels shows corresponding levels of βactin mRNA as control.

FIG. 13. Western blot analysis of FMDV 3D (Panel A) and VP1 (Panel B) proteins expressed in tTA LF-BK and Entpd6 cells clone 30. Cytoplasmic cell extracts were separated by SDS-PAGE, blotted and viral proteins identified with 3D and VP1 antisera, respectively (upper panels). α-tubulin was used as loading control (lower panels). 3CD indicates the precursor of 3D.

FIG. 14. Immunofluorescence assay of tTA and Entpd6 cells clone 30 infected with FMDV O/UK/2001 at 4 hpi, infected at MOI 10 (upper panels A and B, respectively) or at MOI 0.1 (lower panels C and D, respectively) using Mabs against the structural viral proteins. Red, Alexa Fluor 594 goat anti-mouse IgG. (Molecular Probes).

FIG. 15 provides Table 6, which is a table of genes identified as being associated with FMDV susceptibility.

### DETAILED DESCRIPTION

Methods and compositions for identifying CGEPs required for viral infection of mammalian cells are provided. Also provided are methods of inhibiting viral infection of mammalian cells by inhibiting the activity of one or more CGEPs (e.g., as identified in accordance with methods of the invention) in the cells. Aspects of the invention further include specifically identified CGEPs implicated in mammalian cell infection of specific viruses, e.g., African Swine Fever Virus and Foot and Mouth Disease Virus, and methods of modulating their activity, e.g., to achieve viral resistance.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing various aspects of the subject invention, methods of identifying CGEPs are reviewed first in greater detail. Next, specific CGEPs involved susceptibility of mammalian cells to infection by ASFV and FMDV are reviewed, as well as methods for modulating their activity.

As summarized above, aspects of the invention include methods of identifying “cellular genes exploited by pathogens” (i.e., CGEPs), particularly in mammalian cells. GGEPs are cellular genes that encode products that are involved in some manner in viral infection of a cell, e.g., involved in replication of the virus in the cell, as reviewed in the background section above.

Methods practiced in accordance with the invention can rapidly identify CGEPs in mammalian cells. Mammalian cells of interest include, but are not limited to: ungulate cells; rodent cells, such as mouse cells, rat cells; primate cells, e.g., human cells; and the like. In certain embodiments, ungulate cells are of interest. The term “ungulate” is used to mean any species or subspecies of porcine (pig), bovine (cattle), ovine (sheep) and caprine (goats). In general the term encompasses hooved domestic or farm animals. The terms “porcine” and “pig” are used interchangeably herein and refer to any porcine species and/or subspecies of porcine and the same meaning applies as to cows, sheep and goats.

Methods practiced in accordance with the invention may employ assays which include homozygous functional inactivation of chromosomal genes in mammalian cells coupled with screens that identify cell clones that consequently acquire (from the homozygous functional inactivation) phenotypic properties of interest, e.g., resistance to infection by a virus of interest. In the homozygous functional inactivation step of the methods, chromosomal genes in a mammalian cellular library are homozygously functionally inactivated, such that the activity of the genes, e.g., as embodied in the activity of the protein encoded by the genes, is inhibited. As the chromosomal genes are homozygously inactivated, the methods may be viewed as methods of providing pan-allelic inhibition of a gene. In this step of embodiments of the invention, a population of cells, e.g., in the form of a cell culture (as described below in greater detail) is contacted with a Random Homozygous Knock Out (i.e., RHKO) library. In certain of these embodiments, the methods include the production of antisense RNA from members of the RHKO library which operates to provide pan-allelic inhibition of the gene. By “pan-allelic” it is intended that all genes complementary to the antisense sequence are inactivated, e.g., in that the mRNA will bind to the sequence and be degraded or be otherwise rendered to a state of non-function or lessened function, such that the genes are inhibited from expressing proteins, whether the copies are identical or allelic. By inhibiting the function of transcripts encompassing different alleles in different members of cell populations, one can screen for and identify proteins necessary for viral infection, as reviewed in greater detail below.

Any convenient method of achieving the desired homozygous functional inactivation may be employed. Of interest in certain embodiments is the use of an RHKO protocol.

In certain embodiments, the RHKO protocol employed is the RHKO protocol described in U.S. Pat. No. 5,679,523, the disclosure of which protocol is incorporated herein by reference. This RHKO protocol employs a gene search vector (GSV) and therefore may be referred to as an RHKO/GSV protocol. Briefly, for the RHKO/GSV protocol, a construct is employed in a random homozygous knock-out strategy, where the construct includes a GSV for introduction into the host cells. The GSV may be an RNA virus vector, such as MMLV (Moloney murine leukemia virus) etc., where the vector provides for random chromosomal insertion. In certain embodiments, RNA viruses are used that have long terminal repeats that are efficient in substantially randomly inserting into the genome. Other constructs may be employed that will provide for the insertion of the construct into the host genome. A reporter gene may be included, such as the β-geo reporter gene, as desired. The GSV construct lacks a promoter and enhancer in the long terminal repeats of the integrating virus. The expression of the reporter gene is dependent on transcription proceeding into the GSV from the adjoining segment of chromosomal DNA. A splice acceptor site (“SA”) that fuses the reporter gene to chromosomally initiated transcripts is located 5′ to the reporter gene and the antisense promoter is inserted 5′ to the DNA sequence that encodes the SA site in the transcript. At the time of insertion of the provirus into the chromosome, the antisense promoter is turned off. Transcription from this promoter is activated by introduction of a separate transactivation construct or an inducible promoter, such as the tetracycline-controlled promoter. The system is designed so that antisense RNA from the regulated promoter will inactivate transcripts initiated in chromosomal genes that contain the GSV-derived provirus, and concomitantly will inactivate transcripts from other copies of these genes. Clones in which such random homozygous inactivation leads to identifiable phenotypes of interest, e.g., resistance to viral infection, can be isolated from a heterogeneous cell population. As one copy of the gene containing GSV is inactivated by the GSV insertion itself, inactivation of only one additional copy, by an antisense mechanism is required for a phenotypic effect. The effectiveness of antisense knockout may be monitored, as desired, by reduced expression of the reporter. The antisense promoter in the provirus can be turned off again by removing the gene encoding the transactivator protein from the cell or by adding tetracycline (for Tet-off constructs).

In certain embodiments, the RHKO protocol employed is the RHKO protocol described in PCT published application no. WO 2005/074511, the disclosure of which protocol is incorporated herein by reference. This RHKO protocol employs libraries that express nucleic acids of known sequence (e.g., expressed sequence tags (ESTs)) and therefore may be referred to as an RHKO/EST protocol. Briefly, in certain embodiments RHKO/EST protocols, an RNA viral vector, e.g. lentiviral vector, is employed. The viral vector conveniently comprises a selection gene, e.g. antibiotic resistance, in an orientation opposite to the viral transcription, an inducible transcriptional control region, e.g. a Tet responsive element in conjunction with a promoter, and a multiple cloning site for insertion of ESTs, with these components placed between the long terminal repeats of the viral vector.

Where RHKO protocols are employed, such as those protocols reviewed above, an RHKO library, e.g., as described above and in the cited patent publications herein incorporated by reference, is contacted with a cellular population under appropriate conditions such that each member of the library is introduced into a member of the cellular population.

With RHKO/GSV, it is found that infection of 10⁶ to 5×10⁶ cells by GSV retrovirus results in at least 10⁵ independent knockout events in transcriptionally active genes. Multiple independent pools of GSV-infected cells may be carried through the RHKO protocol in parallel to ensure that the collection of libraries has a high likelihood of containing insertional events in a complete set of genes. cDNA cloning of transcripts containing chromosomally-encoded sequences fused to a reporter gene in the GSV may be carried out for novel chromosomal sequences identified by 5′ RACE cloning. Sequencing of these cDNAs may in some instances identify a previously known gene discovered earlier in another context, whereas in other instances, the gene may be novel. Southern blot analysis may be done to confirm that the cDNA obtained represents the chromosomal locus containing the GSV. RNA isolated from cells in which CGEPs are inactivated by RHKO may be used directly for the microarray experiments described below.

In the case of RHKO/EST protocols, in certain embodiments large libraries are employed, where these libraries may or may not have some redundancy. In certain embodiments, the libraries may include at least about 10,000 ESTs, such as at least about 20,000 ESTs, fragments of about 5,000 or more genes, such as of about 10,000 or more genes. In certain embodiments, the libraries have about 25,000 or more ESTs, such as about 35,000 or more ESTs, where about 15,000 or more genes, such as about 20,000 or more genes, may be represented.

The library may be introduced into the target cell population using any convenient protocol. For example, the constructs may be introduced by retroviral infection, electroporation, fusion, polybrene, lipofection, calcium phosphate precipitated DNA, or other conventional techniques. Particularly, the construct is introduced by viral infection for largely random integration of the construct in the genome. The construct is introduced into cells by any of the methods described above.

The cells employed in methods in accordance with the invention may be host cells, including primary cells or cell lines, particularly cells of the organ(s) for which the virus is tropic, or other cells that are more convenient for use in the laboratory and provide an appropriate environment can be used, such as cells from an exogenous host. These cells include, but are not limited to: Vero cells, pig kidney (IBRS 2) cells, baby hamster kidney (BHK 21) cells, bovine kidney (BK) cells, IBRS 2 cell lines, pig cell line SK6 as well as human cell lines, e.g., human neuroblastoma cells (SN—K—SH), normal mammary epithelial (M), prostate carcinoma (M21), cervix carcinoma (HeLa), osteosarcoma (TE8J), melanoma (HT144 and A375P), breast carcinoma (MCF7, MDA-MB435 and MDA468TA3), and ovarian carcinoma YKT2 and OC314; etc. The cells may be grown and maintained under conventional conditions, such as those reported in the experimental section, below.

The cells of the resultant cellular library, e.g., produced as described above, are then assayed (i.e., screened or evaluated) for a cell phenotype of interest, e.g., a cell phenotype distinguishable from the wild-type phenotype. In accordance the present invention, the phenotype of interest is resistance to infection by a virus. In certain embodiments, the phenotype of interest is resistance to viral infection, where the resistance to viral infection is conferred on the cell by inactivation of one or more CGEP genes. In certain embodiments, the inactivated GGEP genes are genes that are involved in viral replication within the cell. As such, the altered cell phenotype of interest may be a change from the ability of a cell to support the propagation of, or be subject to the pathogenic effects of viruses to resistance to infection, propagation, or pathogenicity of viruses.

The cells may be screened for the phenotype of interest using any convenient protocol. In methods of the invention, the cells are challenged one or more times with a virus of interest, and those cells that exhibit a resistant phenotype are then identified for further evaluation, e.g., to identify one or more CGEPs that confer the virally resistant phenotype of interest. In this step of the invention, the cells are contacted with a virus of interest under conditions sufficient for the virus to infect the cell if the cell is permissive of infection.

The virus of interest which is employed to challenge the cell in this step of the methods may vary widely, and is chosen primarily with respect to the nature of the CGEPs that are to be identified. Any virus is of interest, such as mammalian viruses, include mammalian viruses that infect domestic animals, laboratory animals and primates, including humans, may be employed by methods of the invention. Illustrative of viruses of interest are, without limitation: adenovirus, African swine fever virus, bovine calicivirus, bovine enteric coronavrus, canine parvovirus, Ebola virus, hantavirus, hepatitis B virus, herpesvirus, influenza virus, mammalian reovirus, papilloma virus, paramyxovirus, rotavirus, etc. There are also viruses that affect birds, such as chickens and other avian species that may be of interest. In addition, the subject method can be used with plant viruses. The viruses of primary interest are those that result in fatalities, have substantial economic impact, may be used in terrorist attacks etc.

In certain embodiments, the virus is double-stranded DNA virus, and particularly a double-stranded DNA virus that has no RNA stage, where such viruses include, but are not limited to: Adenoviridae, Ascoviridae, Asfarviridae, Baculoviridae, Caudovirales, Fuselloviridae, Herpesviridae, Iridoviridae, Papillomaviridae, Poxviridae, etc. In certain embodiments, the virus of interest is an Asfarviridae, such as African swine fever virus, e.g., African swine fever virus (isolate Malawi LIL 20/1); African swine fever virus (strain BA71V); African swine fever virus (strain E-70/isolate MS44); African swine fever virus (strain E-75); African swine fever virus (strain LIS57); African swine fever virus isolate CHIREDZI/83/1/CH1; African swine fever virus isolate crocodile/96/1/CR1; African swine fever virus isolate crocodile/96/3/CR3; African swine fever virus isolate Haiti 811; African swine fever virus isolate Lisbon 60/lis60; African swine fever virus isolate pretoriuskop/96/5/pr5; African swine fever virus isolate wildebeeslaagte/96/1/m1; and African swine fever virus strain E-70/isolate MS16.

In certain embodiments, the virus is single-stranded RNA virus, and particularly a ssRNA virus that has no DNA stage, where such viruses include, but are not limited to: Astroviridae, Caliciviridae, Dicistroviridae, Flaviviridae, Picornaviridae, and the like. In certain embodiments, the virus of interest is a Picornaviridae, such as a Aphthovirus, Cardiovirus, Enterovirus, Erbovirus, Kobuvirus, Parechovirus, Rhinovirus, Teschovirus, etc. In certain embodiments, the virus is an Aphthovirus, e.g., Equine rhinitis A virus or a Foot-and-mouth disease virus (FMDV), e.g., Foot-and-mouth disease virus—type A, Foot-and-mouth disease virus—type Asia 1, Foot-and-mouth disease virus—type C, Foot-and-mouth disease virus—type O, Foot-and-mouth disease virus—type SAT 1, Foot-and-mouth disease virus—type SAT 2, Foot-and-mouth disease virus—type SAT 3, as well as unclassified Foot-and-mouth disease virus.

In challenging the cells with the virus of interest, infection conditions may be optimized to minimize nonspecific cell survival. Infection times may vary, such as 2 to 5 days of virus exposure with an initial MOI=10. Virus may then be removed and replaced with fresh media and incubated for from 4 to 10, such as about 7 days, with fresh media change at every other day. Cultures may then be re-challenged with virus as above, where this process may include trypsinizing the cells and replating with pooling. Viral titers may then be determined in conventional ways.

Cell cultures infected with the virus of interest, such as, African Swine Fever Virus (ASFV) and Foot-and-Mouth Disease Virus (FMDV) are monitored for viral-induced cytopathology and for outgrowth of virus-resistant cells. Putative resistant cell clones are then identified for further evaluation, e.g., where the resistant cells are picked, cloned by limiting dilution, tested for viral nucleic acid by PCR or RT-PCR, and expanded. The level of host cell restriction for viral infection may be determined using standard virus binding and internalization assays and by monitoring temporal viral gene/protein expression and viral genome replication in resistant cells. Infected lines can be examined ultrastructurally for evidence of infection-induced cellular changes, and specific phenotypic changes beyond simple viral resistance may be correlated with specific gene inactivations. Cell lines or cellular RNAs may be tested for contaminating viral nucleic acid by PCR or RT-PCR, and safety tested by animal inoculation prior to identification and characterization of genes of interest.

After identifying a cell in the library having a change in phenotype of interest, e.g., a virus resistant phenotype, and ascribing the change to the introduced nucleic acid library member therein, such as to the region knocked out or silenced by antisense RNA encoded by the library member present in the cell, the silenced region may be characterized as desired, e.g., the region may be sequenced, the coding region may be used in the sense direction and a polypeptide sequence obtained. The resulting peptide may then be used for the production of antibodies to isolate the particular protein. Also, the peptide may be sequenced and the peptide sequence compared with known peptide sequences to determine any homologies with other known polypeptides. Various techniques may be used for identification of the gene at the locus and the protein expressed by the gene, since the subject methodology provides for a marker at the locus, obtaining a sequence which can be used as a probe and, in some instances, for expression of a protein fragment for production of antibodies. If desired the protein may be prepared and purified for further characterization.

The above described representative random homozygous gene inactivation applications find use in the identification of a genomic coding sequence of interest (i.e., a CGEP) whose lack of expression resulting from the antisense mediated gene inactivation results in a virus resistant phenotype of interest, as described above.

As such, the subject methods find use in the identification of CGEPs for viruses of interest, particularly mammalian CGEPs for viruses of interest, such as the viruses reviewed above. Using the above protocols, mammalian CGEPs can be readily identified for a given virus of interest.

The identification of CGEPs in animal model systems provides the opportunity to correlate natural variation in animal populations with insusceptibility to a virus, or where the CGEPs are required in common, potentially to a wider range of viruses. This is followed by the isolation of animal homologs and a search for polymorphisms in these genes. Standard methods can be used. PCR products representing CGEPs are sequenced to identify polymorphisms.

The identified proteins expressed by the CGEPs identified in accordance with the invention can be used to identify naturally-occurring or induced polymorphisms that provide for resistance to viral infection. The proteins can also be employed in identifying compounds that can interfere with the mechanism associated with the interaction of the virus and the endogenous protein for infection. As such, the proteins encoded by identified CGEPs that are therefore identified as involved with viral infection and propagation can be used in a number of ways. As indicated previously, one can screen cells from various members of the host species to determine whether the particular animal has a polymorphism in the protein that inhibits viral infection. Alternatively, one may modify the protein using recombinant techniques to identify a polymorphism that provides protection from viral infection. The protein may be used as a target for identifying compounds that inhibit viral infection by first determining if the compound binds to the protein. This test is then followed by determining whether the compound provides protection from viral infection.

In certain embodiments, CGEPs, such as those identified using methods in accordance with the invention as described above, are inactivated in a cell or host, e.g., to confer viral resistance on the host. As such, methods of inactivating CGEPs in a cell or host to confer viral resistance to the host are provided by the invention. CGEP inactivation may be achieved in a number of different ways, where illustrative protocols for achieving CGEP inactivation are reviewed in greater detail below in connection with the sections of the application that provide review of specific mammalian CGEPs identified as important in African Swine Fever Virus (ASFV) infection and Foot-and-Mouth Disease Virus (FMDV) infection.

### Identification of African Swine Fever Virus (ASFV) CGEPs

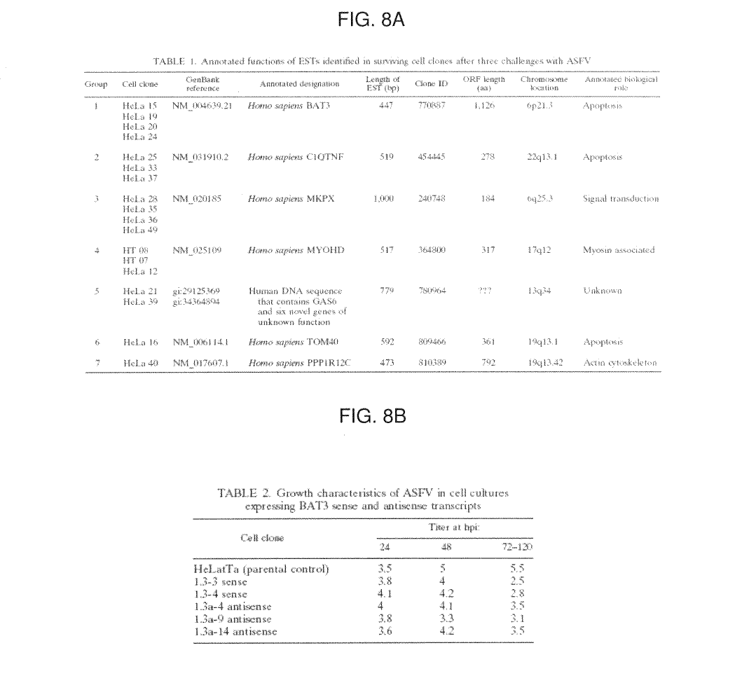

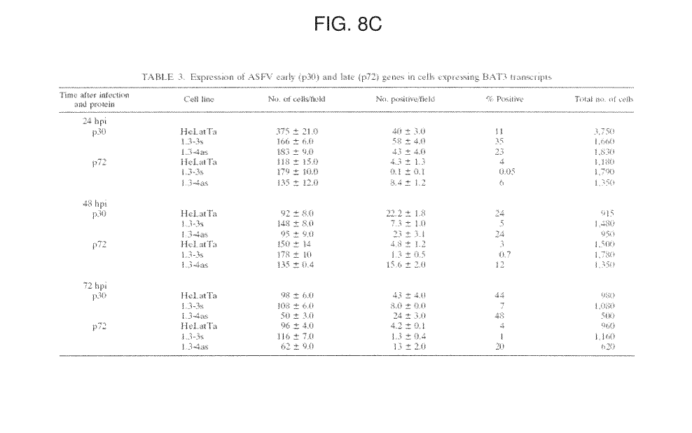

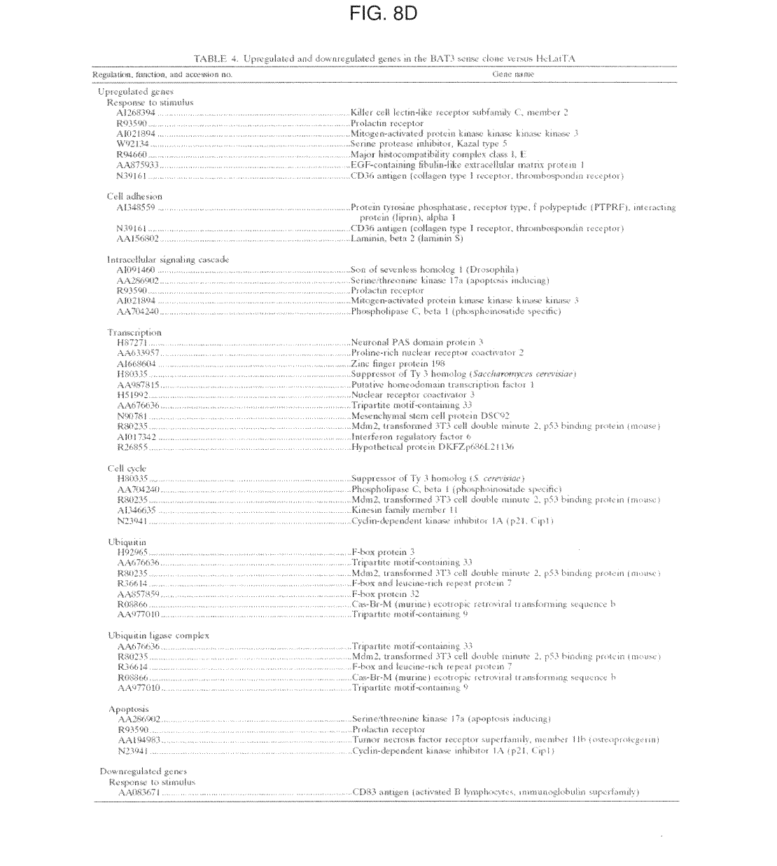

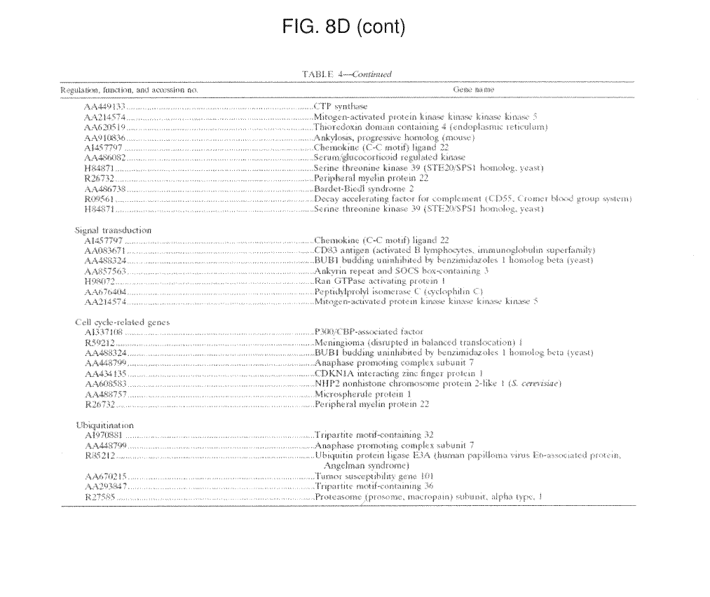

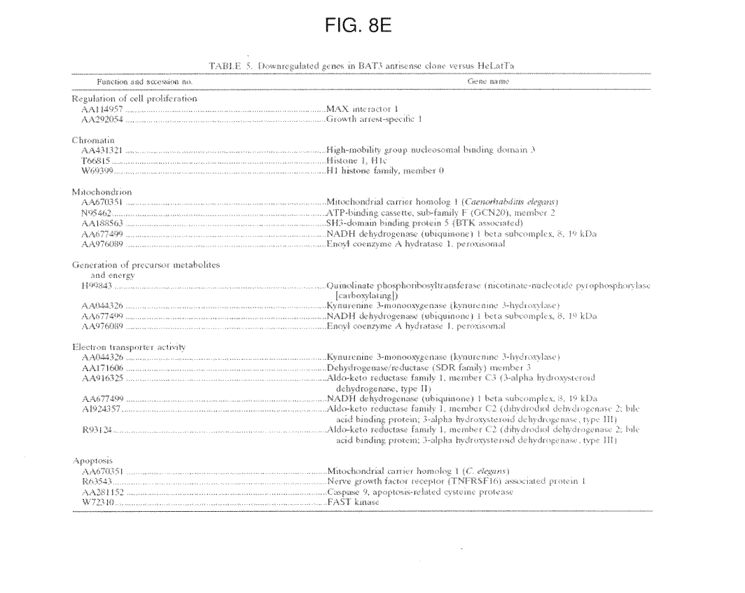

Exemplifying the power of the methods described above is the identification of a number of ASFV CGEPs, where these CGEP genes are: BAT3 (e.g., Homo sapiens BAT3 (GenBank Ref. NM_{—}004639.21)); C1QTNF (e.g., Homo sapiens C1QTNF (GenBank Ref: NM_{—}031910.2); MKPX (e.g., Homo sapiens MKPX (GenBank Ref: NM_{—}020185); MYOHD1 (e.g., Homo sapiens MYOHD1 (GenBank Ref: NM_{—}025109); TOMM40 (e.g., NM_{—}006114.1); and PP1R12C (e.g., Homo sapiens NM_{—}017607); as well as the genes present in an identified region having GenBank Ref. Nos. gi:29125369 and gi:34364894 which contains six novel genes of previously unidentified function. Further details about illustrative ASFV CGEPs are provided in Table 1 in FIG. 8A. Of interest are these particular ASFV CGEPs, as well as homologues thereof, such as mammalian homologues thereof, including ungulate homologues thereof, e.g., swine homologues thereof. By homologue is meant a protein having at least about 35%, such as at least about 40% and including at least about 60% amino acid sequence identity to the specific human proteins as identified above (e.g., as as measured by the BLAST compare two sequences program available on the NCBI website using default settings).

### Identification of Foot-and-Mouth Disease Virus (FMDV) CGEPs

Also exemplifying the power of the methods described above is the identification of a number of FMDV CGEPs, where these FMDV genes are: ectonucleoside triphosphate diphosphohydrolase 6 (Entpd6) (e.g., Homo sapiens NTPDase 6 (GenBank Ref. No. NM_{—}001776)); interferon regulatory factor 7 (e.g., Homo sapiens IRF 7 (GenBank Ref. No. NM_{—}004031); Homo sapiens, Similar to histocompatibility 13, clone MGC (Accession number BC008959); Homo sapiens hypothetical protein FLJ21918 (accession number NM_{—}024939); Soares fetal liver spleen 1NFLS S1 Homo sapiens cDNA (accession number R88919); NIH-MGC-97 Homo sapiens cDNA (accession number BG720581); the Homo sapiens gene having assigned accession number AL953229; and the like. As such, CGEPs of interest in FMDV include, but are not limited to: Homo sapiens ectonucleoside, Homo sapiens interferon (IRF7), Homo sapiens hypothetical protein, Unconventional Myosin IX, Homo sapiens cDNA similar to RI KEN cDNA BC014601, HTB Chromosome 8, Homo sapiens cDNA NIH_MGC_{—}97, Glycoprotein hormone G-protein coupled receptor, Protein-amino acid transporter (Soares testis NHT), Homo sapiens PL6 protein, Soares fetal liver spleen, MAGE resequences, MAGF homo sapiens cDNA, Homo sapiens dopa decarboxylase, Sorares fetal liver spleen 1 NFLS, Soares fetal heart, Homo sapiens succinate dehydrogenase complex. See also Table 6, FIG. 15 for additional FMDV CGEPs of interest. Of interest are these particular FMDV CGEPs, as well as homologues thereof, such as mammalian homologues thereof, including ungulate homologues thereof. By homologue is meant a protein having at least about 35%, such as at least about 40% and including at least about 60% amino acid sequence identity to the specific human proteins as identified above (e.g., as as measured by the BLAST compare two sequences program available on the NCBI website using default settings).

### Utility

The different CGEP genes identified in accordance with the invention may be used individually or together providing host animals resistant to the viral diseases. Cells from the animals, e.g. swine and bovine, may be screened for viral resistance and the specific genes isolated, amplified and sequenced for determining polymorphisms. These animals may then be bred to produce virally resistant animals. Virally resistant animals are animals that, because of the presence of modified CGEP (as compared to wild type) are resistant to viral infection in some manner, where resistant may be identified in a number of different ways, such as reduced or even non-existent levels of viral replication (determined by assessing viral titer), etc.

Alternatively, susceptible hosts may be modified to express the resistant allele of the gene. One can employ animal embryos that are modified by knockout of the endogenous susceptible gene and knockin of the exogenous resistant gene. Alternatively, the resistant gene may be used to replace the susceptible gene, by homologous recombination, introducing the polymorphism into the endogenous gene. The embryos may then be implanted into pseudopregnant females and allowed to grow to term. The resulting progeny may then be cross-bred to homozygosity to provide virus resistant hosts. Alternatively, one may genetically modify nuclei for displacement of the nucleus in an oocyte and implant the oocyte in a pseudopregnant host. Any convenient protocol may be employed, including but not limited to those transgenic animal production protocols described in U.S. Pat. Nos. 6,673,987 and 5,994,610; as well as WO99/01164, the disclosures of which protocols are herein incorporated by reference.

One may also use the resistant polymorphic proteins for screening of domestic animals for the viral resistance genes. Cells and/or blood may be taken from the animal and used as a source for protein and/or nucleic acid. Any convenient method of screening, such as immunoassays, isolation of DNA, amplification with PCR and screening with complementary sequences, or the like, can be employed. There are a large number of commercially available techniques that can be modified to be applied for the present purpose. Those animals homozygous for the resistant gene may be then be mated and propagated, while heterozygous animals may be mated to homozygosity and the resultant homozygous progeny expanded and propagated.

By identifying proteins that cooperate with viruses for infection and/or propagation, the identified proteins can be used in a number of different ways. One can screen the same or different species susceptible to the virus to identify individuals that are resistant to the virus. One then isolates the gene to determine whether the resulting protein is polymorphic with the protein that allows for susceptibility. Once a resistant polymorph is identified, the resistant strain may be crossed with other strains of the same species and then cross-bred to provide for homozygosity of the polymorph. Alternatively, embryonic stem cells can be modified by homologous recombination to change the susceptible gene to the resistant gene.

Instead of screening for host cells from members of the species having resistance to the virus as a result of an identified gene, one may randomly mutate the gene using mutagenic agents and then transfer the mutated genes into host cells of the susceptible species or surrogate cells in which the gene is not expressed. The cells would then be screened with the virus for susceptibility and those polymorphs further tested for their protective effect in host cells and used to genetically modify embryonic cells to establish the viability of the host and its resistance to the virus.

### Modulation of CGEP Activity

The inhibition of expression or activity of one or more CGEPs results in a virally resistant phenotype. Therefore, the gene(s) may be used in a variety of ways. The gene(s) can be used for the expression and production of the encoded protein to identify agents which inhibit the encoded protein to determine the role that encoded protein plays in the virally resistant phenotype. The encoded protein may be used to produce antibodies, antisera or monoclonal antibodies, for assaying for the presence of encoded protein in cells. The DNA sequences may be used to determine the level of mRNA in cells to determine the level of transcription. In addition, the gene may be used to isolate the 5′ non-coding region to obtain the transcriptional regulatory sequences associated with expression of the encoded protein. By providing for an expression construct which includes a marker gene under the transcriptional control of the encoded protein transcriptional initiation region, one can follow the circumstances under which an encoded protein is turned on and off.

Fragments of the CGEP gene of interest may be used to identify other genes having homologous sequences using low stringency hybridization and the same and analogous genes from other species, such as primate, particularly human, and the like.

The CGEP gene or fragments thereof may be introduced into an expression cassette for expression or production of antisense sequences, where the expression cassette may include upstream and downstream in the direction of transcription, a transcriptional and translational initiation region, the CGEP gene, followed by the translational and transcriptional termination region, where the regions will be functional in the expression host cells. The transcriptional region may be native or foreign to the CGEP gene, depending on the purpose of the expression cassette and the expression host. The expression cassette may be part of a vector, which may include sites for integration into a genome, e.g., LTRs, homologous sequences to host genomic DNA, etc., an origin for extrachromosomal maintenance, or other functional sequences.

The methods find use in a variety of therapeutic applications in which it is desired to modulate, e.g., increase or decrease, CGFP expression/activity in a target cell or collection of cells, where the collection of cells may be a whole animal or portion thereof, e.g., tissue, organ, etc. As such, the target cell(s) may be a host animal or portion thereof, or may be a therapeutic cell (or cells) which is to be introduced into a multicellular organism, e.g., a cell employed in gene therapy. In such methods, an effective amount of an active agent that modulates CGEP expression and/or activity, e.g., enhances or decreases CGEP expression and/or activity as desired, is administered to the target cell or cells, e.g., by contacting the cells with the agent, by administering the agent to the animal, etc. By effective amount is meant a dosage sufficient to modulate CGEP expression in the target cell(s), as desired.

In the subject methods, the active agent(s) may be administered to the targeted cells using any convenient means capable of resulting in the desired modulation of CGEP expression and/or activity. Thus, the agent can be incorporated into a variety of formulations, e.g., pharmaceutically acceptable vehicles, for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments (e.g., skin creams), solutions, suppositories, injections, inhalants and aerosols. As such, administration of the agents can be achieved in various ways, including oral, buccal, rectal, parenteral, intraperitoneal, intradermal, transdermal, intracheal, etc., administration.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Where the agent is a polypeptide, polynucleotide, analog or mimetic thereof, e.g. oligonucleotide decoy, it may be introduced into tissues or host cells by any number of routes, including viral infection, microinjection, or fusion of vesicles. Jet injection may also be used for intramuscular administration, as described by Furth et al. (1992), Anal Biochem 205:365-368. The DNA may be coated onto gold microparticles, and delivered intradermally by a particle bombardment device, or “gene gun” as described in the literature (see, for example, Tang et al. (1992), Nature 356:152-154), where gold microprojectiles are coated with the DNA, then bombarded into skin cells. For nucleic acid therapeutic agents, a number of different delivery vehicles find use, including viral and non-viral vector systems, as are known in the art.

Those of skill in the art will readily appreciate that dose levels can vary as a function of the specific compound, the nature of the delivery vehicle, and the like. Preferred dosages for a given compound are readily determinable by those of skill in the art by a variety of means.

The subject methods find use in the treatment of a variety of different conditions in which the modulation, e.g., enhancement or decrease, of CGEP expression and/or activity in the host is desired. By treatment is meant that at least an amelioration of the symptoms associated with the condition afflicting the host is achieved, where amelioration is used in a broad sense to refer to at least a reduction in the magnitude of a parameter, e.g. symptom (, associated with the condition being treated. As such, treatment also includes situations where the pathological condition, or at least symptoms associated therewith, are completely inhibited, e.g. prevented from happening, or stopped, e.g. terminated, such that the host no longer suffers from the condition, or at least the symptoms that characterize the condition.

A variety of hosts are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

In certain embodiments, the methods of CGEP modulation are methods of inhibiting CGEP activity. Such methods find use in, among other applications, the treatment and/or prevention of viral related complications, and analogous disease conditions. In these methods, modulation, e.g., inhibition of CGEP expression/activity may be accomplished using a number of different types of agents.

In certain embodiments, naturally occurring or synthetic small molecule compounds of interest include numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Such molecules may be identified, among other ways, by employing the screening protocols described below.

In another method of modifying protein activity, mutant alleles can be expressed in the cell which inhibit the activity in a dominant manner (“dominant negative mutations”). Such dominant negative mutants can act, inter alia, by flooding the cell with an inactive form of the protein which nevertheless binds the natural substrate, or by introducing mutant subunits which render a multimeric structure inactive, or by other known means. For example, a mutant subunit with an activity domain deleted but retaining an association domain (as can be formed by partial gene deletions) can form inactive multimeric complexes.

Dominant negative mutations are mutations to endogenous genes or mutant exogenous genes that when expressed in a cell disrupt the activity of a targeted protein species. Depending on the structure and activity of the targeted protein, general rules exist that guide the selection of an appropriate strategy for constructing dominant negative mutations that disrupt activity of that target (Hershkowitz, 1987, Nature 329:219-222). In the case of active monomeric forms, over expression of an inactive form can cause competition for natural substrates or ligands sufficient to significantly reduce net activity of the target protein. Such over expression can be achieved by, for example, associating a promoter of increased activity with the mutant gene. Alternatively, changes to active site residues can be made so that a virtually irreversible association occurs with the target ligand. Such can be achieved with certain tyrosine kinases by careful replacement of active site serine residues (Perlmutter et al., 1996, Current opinion in Immunology 8:285-290).

In the case of active multimeric forms, several strategies can guide selection of a dominant negative mutant. Multimeric activity can be decreased by expression of genes coding exogenous protein fragments that bind to multimeric association domains and prevent multimer formation. Alternatively, overexpression of an inactive protein unit of a particular type can tie up wild-type active units in inactive multimers, and thereby decrease multimeric activity (Nocka et al., 1990, The EMBO J. 9:1805-1813). For example, in the case of dimeric DNA binding proteins, the DNA binding domain can be deleted from the DNA binding unit, or the activation domain deleted from the activation unit. Also, in this case, the DNA binding domain unit can be expressed without the domain causing association with the activation unit. Thereby, DNA binding sites are tied up without any possible activation of expression. In the case where a particular type of unit normally undergoes a conformational change during activity, expression of a rigid unit can inactivate resultant complexes. For a further example, proteins involved in cellular mechanisms, such as cellular motility, the mitotic process, cellular architecture, and so forth, are typically composed of associations of many subunits of a few types. These structures are often highly sensitive to disruption by inclusion of a few monomeric units with structural defects. Such mutant monomers disrupt the relevant protein activities.

In yet other embodiments, expression of the CGEP of interest is inhibited. Inhibition of CGEP expression may be accomplished using any convenient means, including use of an agent that inhibits CGEP expression (e.g., antisense agents, agents that interfere with transcription factor binding to a promoter sequence of the target CGEP gene, etc), inactivation of the CGEP gene, e.g., through recombinant techniques, etc.

For example, antisense molecules can be used to down-regulate expression of the target protein in cells. The anti-sense reagent may be antisense oligodeoxynucleotides (ODN), particularly synthetic ODN having chemical modifications from native nucleic acids, or nucleic acid constructs that express such anti-sense molecules as RNA. The antisense sequence is complementary to the mRNA of the targeted protein, and inhibits expression of the targeted protein. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. It has been found that short oligonucleotides, of from 7 to 8 bases in length, can be strong and selective inhibitors of gene expression (see Wagner et al. (1996), Nature Biotechnol. 14:840-844).

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides may be chemically synthesized by methods known in the art (see Wagner et al. (1993), supra, and Milligan et al., supra.) Preferred oligonucleotides are chemically modified from the native phosphodiester structure, in order to increase their intracellular stability and binding affinity. A number of such modifications have been described in the literature, which alter the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH₂-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The α-anomer of deoxyribose may be used, where the base is inverted with respect to the natural β-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

As an alternative to anti-sense inhibitors, catalytic nucleic acid compounds, e.g. ribozymes, anti-sense conjugates, etc. may be used to inhibit gene expression. Ribozymes may be synthesized in vitro and administered to the patient, or may be encoded on an expression vector, from which the ribozyme is synthesized in the targeted cell (for example, see International patent application WO 9523225, and Beigelman et al. (1995), Nucl. Acids Res. 23:4434-42). Examples of oligonucleotides with catalytic activity are described in WO 9506764. Conjugates of anti-sense ODN with a metal complex, e.g. terpyridylCu(II), capable of mediating mRNA hydrolysis are described in Bashkin et al. (1995), Appl. Biochem. Biotechnol. 54:43-56.

In addition, the transcription level of a CGEP can be regulated by gene silencing using RNAi agents, e.g., double-strand RNA (Sharp (1999) Genes and Development 13: 139-141). RNAi, such as that which employs double-stranded RNA interference (dsRNAi) or small interfering RNA (siRNA), has been extensively documented in the nematode C. elegans (Fire, A., et al, Nature, 391, 806-811, 1998) and routinely used to “knock down” genes in various systems. RNAi agents may be dsRNA or a transcriptional template of the interfering ribonucleic acid which can be used to produce dsRNA in a cell. In these embodiments, the transcriptional template may be a DNA that encodes the interfering ribonucleic acid. Methods and procedures associated with RNAi are also described in WO 03/010180 and WO 01/68836, all of which are incorporated herein by reference. dsRNA can be prepared according to any of a number of methods that are known in the art, including in vitro and in vivo methods, as well as by synthetic chemistry approaches. Examples of such methods include, but are not limited to, the methods described by Sadher et al. (Biochem. Int. 14:1015, 1987); by Bhattacharyya (Nature 343:484, 1990); and by Livache, et al. (U.S. Pat. No. 5,795,715), each of which is incorporated herein by reference in its entirety. Single-stranded RNA can also be produced using a combination of enzymatic and organic synthesis or by total organic synthesis. The use of synthetic chemical methods enable one to introduce desired modified nucleotides or nucleotide analogs into the dsRNA. dsRNA can also be prepared in vivo according to a number of established methods (see, e.g., Sambrook, et al. (1989) Molecular Cloning: A Laboratory Manual, 2nd ed.; Transcription and Translation (B. D. Hames, and S. J. Higgins, Eds., 1984); DNA Cloning, volumes I and II (D. N. Glover, Ed., 1985); and Oligonucleotide Synthesis (M. J. Gait, Ed., 1984, each of which is incorporated herein by reference in its entirety). A number of options can be utilized to deliver the dsRNA into a cell or population of cells such as in a cell culture, tissue, organ or embryo. For instance, RNA can be directly introduced intracellularly. Various physical methods are generally utilized in such instances, such as administration by microinjection (see, e.g., Zernicka-Goetz, et al. (1997) Development 124:1133-1137; and Wianny, et al. (1998) Chromosoma 107: 430-439). Other options for cellular delivery include permeabilizing the cell membrane and electroporation in the presence of the dsRNA, liposome-mediated transfection, or transfection using chemicals such as calcium phosphate. A number of established gene therapy techniques can also be utilized to introduce the dsRNA into a cell. By introducing a viral construct within a viral particle, for instance, one can achieve efficient introduction of an expression construct into the cell and transcription of the RNA encoded by the construct.

In another embodiment, the CGEP gene is inactivated so that it no longer expresses a functional protein. By inactivated is meant that the gene, e.g., coding sequence and/or regulatory elements thereof, is genetically modified so that it no longer expresses functional repressor protein. The alteration or mutation may take a number of different forms, e.g., through deletion of one or more nucleotide residues in the region, through exchange of one or more nucleotide residues in the region, and the like. One means of making such alterations in the coding sequence is by homologous recombination. Methods for generating targeted gene modifications through homologous recombination are known in the art, including those described in: U.S. Pat. Nos. 6,074,853; 5,998,209; 5,998,144; 5,948,653; 5,925,544; 5,830,698; 5,780,296; 5,776,744; 5,721,367; 5,614,396; 5,612,205; the disclosures of which are herein incorporated by reference.

Also provided by the subject invention are screening assays designed to find modulatory agents of CGEP activity, e.g., inhibitors or enhancers of CGEP activity, as well as the agents identified thereby, where such agents may find use in a variety of applications, including as therapeutic agents, as described above. The screening methods may be assays which provide for qualitative/quantitative measurements of CGEP activity in the presence of a particular candidate therapeutic agent. The screening method may be an in vitro or in vivo format, where both formats are readily developed by those of skill in the art. Depending on the particular method, one or more of, usually one of, the components of the screening assay may be labeled, where by labeled is meant that the components comprise a detectable moiety, e.g. a fluorescent or radioactive tag, or a member of a signal producing system, e.g. biotin for binding to an enzyme-streptavidin conjugate in which the enzyme is capable of converting a substrate to a chromogenic product.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used.

A variety of different candidate agents may be screened by the above methods. As reviewed above, candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents may be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Using the above screening methods, a variety of different therapeutic agents may be identified. Such agents may target CGEP itself, or an expression regulator factor thereof. Such agents may be inhibitors or promoters of CGEP activity, where inhibitors are those agents that result in at least a reduction of CGEP activity as compared to a control and enhancers result in at least an increase in CGEP activity as compared to a control. Such agents may be find use in a variety of therapeutic applications, as reviewed above.

### Kits and Systems

Also provided are kits and systems for use in practicing various aspects of the invention. The systems at least include an RHKO library, a cell line and a virus of interest. Kits may include one or more of these components, e.g., present in the same or separate containers.

The kits and systems may also include a number of optional components that find use in the subject methods. Optional components of interest include buffers, reporter enzyme substrates, etc.

In certain embodiments of the subject kits, the kits will further include instructions for practicing the subject methods or means for obtaining the same (e.g., a website URL directing the user to a webpage which provides the instructions), where these instructions are typically printed on a substrate, which substrate may be one or more of: a package insert, the packaging, reagent containers and the like. In the subject kits, the one or more components are present in the same or different containers, as may be convenient or desirable.

The above descriptions are provided so that one of skill in the art may understand how the present invention may be used, and are not intended to be limiting.

The following examples are offered by way of illustration and not by way of limitation.

### Experimental

### I. Identification of Genes Associated With ASFV Susceptibility

### A. Materials and Methods

1. Construction of the EST Expression Vector and Preparation of the HeLatTA and HT144tTA pLentiEST Libraries.

The EST expression construct described in Lu et al, Proc. Nat'l Acad. Sci. USA (2004) 101: 17246-17251, was used. Briefly, a collection of ˜40,000 human sequence-verified ESTs (Invitrogen) was pooled and amplified by PCR using two directional universal primers flanking the EST DNA fragments. The resulting amplified EST products were then digested with NheI and cloned using a modified version of a self-inactivating lentiviral backbone plasmid derived from pRRLsinPPT.CMV.MCS.Wpre (a gift of L. Naldini, HSR-TIGET, Milan, Italy) (Follenzi et al., Nat. Genet. (2000)25:217-222), in which the constitutive cytomegalovirus (CMV) promoter of the original plasmid was replaced with a DNA fragment containing a neomycin resistance expression cassette and a CMV minimal promoter controlled by the tetracycline regulated tetracycline responsive element (TRE) (Gossen et al., Proc. Nat'l Acad. Sci USA (1992) 89:5547-5551). The tetracycline derivative doxycycline (Dox) was used to regulate the promoter in the experiments reported here.

The host cells used in this work, a HeLa human cervical cancer cell line and HT144, a line of human metastatic melanoma cells (Fogh et al., J. Natl. Cancer Inst. (1997) 59:221-226; Gouon et al., Int. J. Cancer (1996) 68: 650-662), were modified to overexpress tetracycline-dependent transcriptional activator (tTA) (Gossen et al., supra) from a pBabetTAPuro retrovirus. pLentiEST libraries were then generated in both HeLatTA and HT144tTA cells as described elsewhere (Lu et al., supra). Briefly, 20 subconfluent 15-cm² plates of HeLatTA cells (ca. 2×10⁷ cells in total) were infected with 500 μl of pLentiEST virus supernatant derived from 293T packaging cells and pseudotyped with the G protein of vesicular stomatitis virus envelope gene in the presence of 4 μg/ml of Polybrene. Following G418 selection (1,000 μg/ml for HeLatTA and 600 μg/ml for HT144 tTa), the G418-resistant clones were pooled in several aliquots and expanded one generation to form the pLentiEST libraries of HeLatTA and HT144tTa, respectively.

### 2. Genomic DNA Extraction and PCR.

Genomic DNA was isolated from 3×10⁶ to 4×10⁶ cultured cells from each clone of interest using the Gentra DNA extraction kit (Gentra Systems), and DNA was dissolved in 100 μl of the hydration buffer included in the kit. The EST DNA fragment in each cell clone was isolated by PCR amplification of the genomic DNA with the universal primers (Lu et al., supra) flanking the EST insert in the viral construct. The amplified PCR products were purified, sequenced, and identified by a BLAST search of the NCBI database. The orientation of the EST in the cell clone was determined by PCR amplification using a Lenti 3′ primer derived from the viral vector and a universal EST forward or EST reverse primer.

### 3. Viruses, Cell Cultures, and Infection.

The ASFV used in these experiments is the Malawi Lil-20/1 isolate (Haresnape et al., Malawi. Epidemiol. Infect. (1988) 101: 173-185), which is a virulent pathogenic African swine fever virus isolated from ticks of the Ornithodoros moubata complex (Ixodoidea: Argasidae) that were collected in the ASF enzootic area of Malawi. The virulent pathogenic virus isolated from swine was adapted to grow in Vero cell cultures by 25 serial passages prior to culture on HeLa or HT144 cells; it also retained virulence properties in swine in vivo (L. Zsak, unpublished data). The replicative cycle of this virus was longer in Vero and HeLa cells than in pig cells and yielded the p30 early ASFV protein in 30% of cells 4 to 8 h after infection (T. Burrage, unpublished data). Progression to late protein was observed at later time points, as indicated below, and death of the entire culture was observed. Cultures of HeLatTA and HT144tTA EST libraries, which did not show any detectable growth alterations compared to the parental cell lines, were infected with the adapted ASFV.

### 4. Titration of ASFV.

Parental HeLatTA cell, HeLatTA cell EST library pools, and individual ASFV-resistant HeLa cell clones were cultured in T75 flasks until they reached 90% confluence. Cells were trypsinized and counted for viability using trypan blue. Cell cultures for growth curve experiments were made in 24-well Primaria plastic plates; cells were plated at a density of 5×10⁶ per well in 10% Dulbecco's modified Eagle's medium (DMEM). For the doxycycline positive cultures, 5 μg per ml Dox was used in the culture medium.

EST libraries and control cells were infected after at least 24 h of growth in culture with ASFV Malawi isolate at the indicated multiplicities of infection (MOIs) and incubated for 2 h at 37° C. Following incubation, cultures were washed three times with prewarmed 10% DMEM, and 1 ml of 10% DMEM was added following the final wash. Samples were collected at various times postinoculation.

Cells were scraped into the medium, and the mixture was transferred into an Eppendorf tube and centrifuged at 3,000 rpm for 5 min. Supernatant was removed, transferred into a new Eppendorf tube, and designated as extracellular virus. Cell pellets were resuspended in 1 ml of fresh medium, freeze-thawed, and sonicated to lyse the cells and disrupt viral aggregates. The “intracellular virus” titers determined from these samples closely paralleled those determined for extracellular virus by testing the supernatants from cultures containing unlysed cells. Two samples were taken at each time point and were stored at ˜70° C. until titration.

Virus titers were determined in primary cultures of swine macrophages. Virus titers were calculated based on the hemadsorption of infected cells and calculated as described previously (Reed et al. Am. J. Hyg. (1938) 27-493-497).

### 5. Dot Blot DNA Hybridization.

Genomic DNA was isolated from the indicated cell lines that had been plated at the same density and then infected at approximately the 80% confluent stage with ASFV (MOI, 5). Duplicate samples were taken at various times postinfection (16 to 72 h), and DNA was extracted from the cell pellet and adjusted to an identical concentration (1 μg per ml). For quantitative hybridization, serial twofold dilutions were made from an initial 100-ng sample and equal amounts of DNA dilutions were transferred onto nitrocellulose membranes. DNA was hybridized with ³²P-labeled ASFV genomic DNA probes using standard procedures.

### 6. Quantitation of Viral Antigen-Containing Cells.

Expression of ASFV early and late genes in BAT3 sense and antisense clones was assayed by fluorescence microscopy of antigens/antibody staining following infection of HeLatTA and BAT3 clones with ASFV. At different times after infection, cell cultures were fixed and immunostained with either anti-p30 (ASFV early protein) or anti-p72 (ASFV late protein). The experiments were performed by counting the viral antigen-containing positive cells in cultures at the different time points. Several thousand cells were observed per time point for each sample.

### 7. Quantitative PCR.

Quantitative PCR experiments for evaluation of cellular BAT3 mRNA production were performed using the Bio-Rad iCycler real-time PCR detection system and the IQ SYBR Green Super Mix kit (catalog no. 170-8880). Oligonucleotide primers were synthesized by the Stanford PAN Facility. The sequences of the forward and reverse probes correspond to exons of the BAT3 sequence available from the National Center for Biotechnology Information. These were as follows: BAT3.1512F, GTGGAACCCGTGGTCATGATGCA (SEQ ID NO:01); BAT31629.F(F2), GTCATGATGCACATGAACATTC (SEQ ID NO:02); BAT3.1634VariantReverse (VR), GGTGGAGCCCAGGGTTTGG (SEQ ID NO:03); BAT3.1743R(R), CCTGCTGTCCCAGGGTTTGG (SEQ ID NO:04). Cell lines were grown until the exponential phase, and total RNA was isolated by using a QIAGEN RNAeasy Plant Mini kit. Five μg of total RNA was used in the synthesis of cDNA using avian myeloblastosis virus reverse transcriptase (Invitrogen) under conditions recommended by the vendor. PCR was performed in a 15-μvreaction mixture volume in the presence of 10 nM forward and reverse primers, 7.5 μl IQ SYBR Green Super mix, and 0.2 μl of cDNA. Each assay consisted of an initial denaturation period of 5 min at 95° C. to activate the polymerase followed by 45 cycles of 95° C. for 15 s, 55° C. for 30 s, and 70° C. for 45 s. Melting curve analysis was used to determine levels of BAT3 transcript produced in the antisense and sense orientations with respect to the control cell line, HeLatTA. The stably expressed GUS β-glucuronidase gene (Aerts et al., Biotechniques (2004) 36:84-91) was used to monitor the input RNA for the three cell lines. Three separate experiments were carried out; the values shown represent the averages from four repeats in each experiment and had a mean standard deviation of <0.5%.

### 8. Microarray Analysis.

The cell clones HeLatTA (parental cell line), BAT3S (dominant negative clone), and BAT3A (BAT3 EST in antisense configuration) were grown to exponential phase, and poly(A) RNA was extracted (Invitrogen PolyA RNA extraction kit) for microarray analysis. The cDNA labeled with Cy3TTP or Cy5TTP was hybridized to human 40K cDNA arrays prepared by the Stanford Functional Genomics Facility. The results were analyzed by the GABRIEL software (Pan et al., Proc. Nat'l Acad. Sci. USA (2002)99:2118-2123) using either pattern-based rules (Pan et al., supra) or a modified t-score algorithm (Troyanskaya et al., Bioinformatics (2001) 17:520-525). Missing values in the microarray data set were estimated with KNNimpute using 14 neighbors (Troyanskaya, supra).

### 9. MTT Viability Assay for Apoptotic Potential.

Cells were grown in 96-well plates to a density of 10,000 cells per well and treated with different dosages of staurosporine to induce apoptosis (Boix et al., Neuropharmacology (1997) 36: 811-821). After 24 h of treatment, cell viability was measured by the 3-(4,5-dimethylthiozol-2-yl)-2,5-diphenyltetrazolium bromide (MTT) assay (Mossman, J. Immunol. Methods (1983) 65: 55-63). The percentage of surviving cells was determined by comparing these values with those obtained for untreated control cells.

### B. Results

### 1. Construction of ASFV-Infected HeLa and HT144 Cell Libraries Expressing ESTs and Isolation of ASFV-Resistant Cell Clones.

Whereas swine macrophages are the natural target cells for ASFV, laboratory strains of the virus have been adapted for replication in a variety of permanent cell lines derived from multiple sources, including primates, where extensive investigations of ASFV molecular biology have been carried out; these cell lines include Vero cells (Alfonso et al., Proteomics (2004)4:2037-2046), monkey kidney MS cells (Santurde et al., Arch. Virol. (1988)117-122), Jurkat cells (Granja et al., J. Immunol. (2006) 176:451-462), and the human myeloid leukemia cell line K562. The human cervical carcinoma (HeLa) and melanoma (HY144) cell lines, which we previously had shown to enable ASFV replication at a rate similar to that observed in Vero cells (L. Zsak, unpublished data) and which were also highly infectible by our lentivirus EST libraries, were chosen as targets for the isolation of cellular genes required for the propagation of ASFV. We infected these cell lines with an ASFV isolate that had been previously adapted to passage in Vero cells but which also remained virulent in swine (see Materials and Methods). The rate of virus replication we observed for this isolate in HeLa and HT144 cells was similar to that observed for Vero cells but was slower than replication in swine macrophages (Zsak, unpublished data).

A previously described pLenti EST library (Lu et al., supra) that expresses a collection of ESTs from a promoter controlled by the TRE (Gossen et al., supra) was introduced into HeLa and HT144 cells that contain the tTA (Gossen et al., supra), which enables regulation of expression of inserted ESTs by the tetracycline derivative doxycycline. Pools of library cells constructed as described in Materials and Methods were grown for one generation before being infected with ASFV at MOIs ranging from 1 to 10. Clones of surviving cells were recultured and challenged through three additional cycles of ASFV infection at a multiplicity of 10, at which time no surviving cells were detected in ASFV-infected cultures of HeLatTA or H144tTA cells that had not received the EST library. PCR amplification and sequence analysis of the EST inserts (see Materials and Methods) identified 18 ESTs corresponding to seven previously annotated genes, shown in Table 1 (See FIG. 8A). Importantly, in some instances multiple clones derived independently from different EST library pools and, in the case of MyoD, ASFV-resistant cell clones derived from different kinds of host cell lines (i.e., both HeLa and HT44), contained the same EST insert.

Two of the ESTs identified in ASFV-resistant HeLa clones contained coding sequences of peptides annotated as being involved in pathways related to apoptosis: BAT3 (Banerji et al., Proc. Nat'l Acad. Sci. USA (1990) 87:2374-2378) and C1q complement, tumor necrosis factor-related protein 6 (C1qTNFR6) (Sanchez-Cordon et al., J. Comp. Pathol. (2002) 127:239-248). The BAT3 gene, which encodes a nuclear protein, previously was mapped to the major histocompatibility complex class III (MHC III) region of human chromosome 6 (Banerji et al.), where it is located between the MHC class I and class II regions. C1qTNFR6, a protein of 278 amino acids, contains a small globular N-terminal domain, a collagen-like Gly/Pro-rich central region, and a conserved C-terminal region, the C1q domain, and is part of the C1 enzyme complex. C1q complement protein is involved in binding of virus (Thielens et al., Immunobiology (2002) 205:563-574) and recognition of microbial surfaces (Shapiro et al., Curr. Biol. (1998)8:335-338).

An EST identified in another one of the clones we isolated (Table 1) (FIG. 8A) encodes the channel-forming subunit of TOM40, a translocase present in the outer membrane of mitochondria (Gabriel et al., EMBO J. (2003) 22:2380-2386; Rappaport, Trends Biochem. Sci. (2002)27-191-197). TOM40 is essential for processing of apocytochrome c to cytochrome c and for protein import into mitochondria (Diekert et al., EMBO J. (2001)20:5626-5634). Two other ESTs, mitogen-activated protein kinase phosphatase (MKPX) and protein phosphatase 1 regulatory subunit 12C (PP1R12C), are related to known signal transduction pathways (Alonso et al., J. Biol. Chem. (2002)277:5524-5528; Tan et al., J. Biol. Chem. (2001)276:21209-21216).

cDNAs representing five of the seven ESTs we identified by this screen were present also in a porcine EST library (NCBI accession number CB483054) (Afonso et al., J. Virol. (2004)78:1858-1864) prepared from the primary cultures of porcine macrophages, and all of these human ESTs had high nucleotide sequence homologies (indicated in parentheses) with their swine counterparts: C1QTNFR6 (88%), MKPX (92%), TOM40 (93%), PP1R12C (93%), and BAT3 (93%).

Experiments using the identified ESTs to reconstitute the ASFV resistance phenotype in naïve cells were carried out in order to confirm that the observed survival to repeated challenges of ASFV infection resulted from the EST sequences.

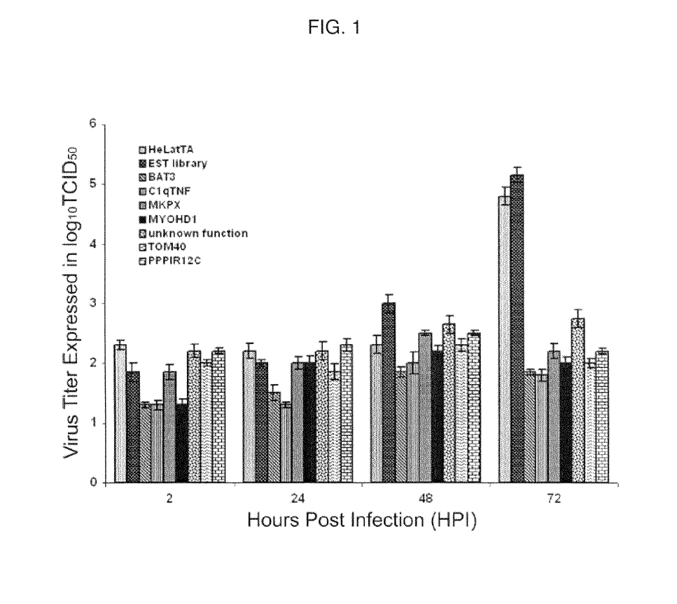

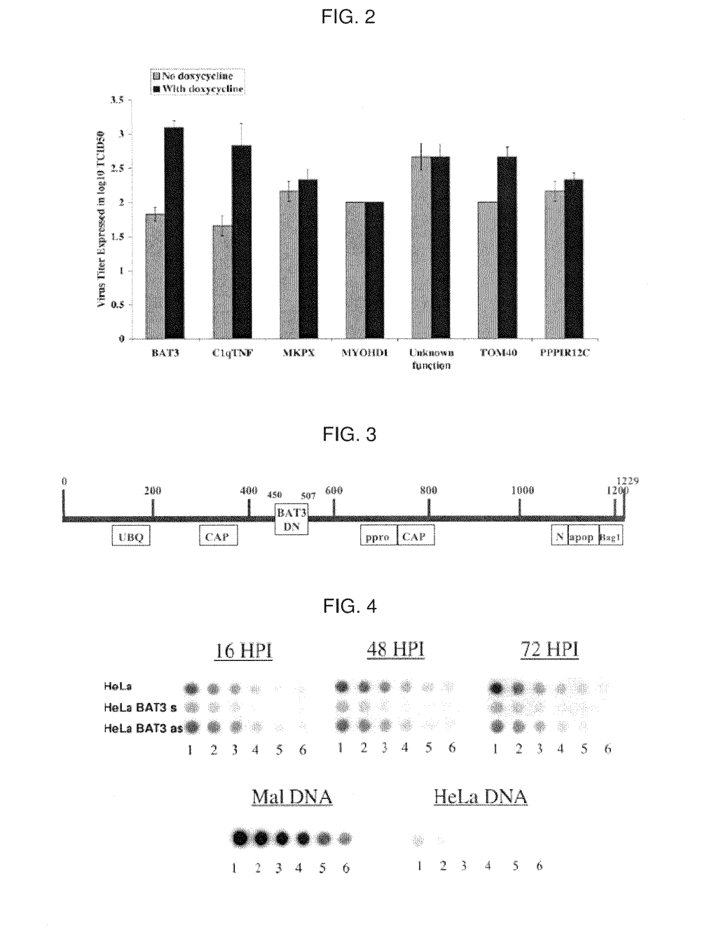

Lentiviral constructs expressing ESTs of each of the seven identified genes from a promoter regulated by the TRE were introduced by transfection into 293 cells and harvested as pLentiEST virus stock mixtures. These were then used to infect the naïve HeLatTA parental cell line, and 10 randomly selected reconstituted clones containing pLentiEST insertions at different chromosomal locations were isolated for each of the seven genes and tested individually for ASFV production following infection by ASFV. A dramatic decrease in virus production (2 to 4 logs) was observed relative to control cultures of parental HeLatTA cells and the HeLatTA EST library pool for each of the tested clones (FIG. 1), and the decrease was partially reversed by the addition of doxycycline to the medium used to culture cells containing the BAT3, C1qTNFR6, and TOM40 EST constructs (FIG. 2). While the ASFV resistance phenotype was reconstituted in naïve cells for the other genes identified by our initial screen, phenotypic reversal by doxycycline was not observed.

The human BAT3 gene, which encodes the BAT3 EST identified here, is shown diagrammatically in FIG. 3. The gene is estimated to specify a protein of 120 kDa that contains several structural domains of possible relevance to the phenotypic properties we observed. The N-terminal portion of the BAT3 protein (amino acids 120 to 190) contains a ubiquitin-like region, a central segment that includes a polyproline-rich stretch, and a C-terminal region that includes both a nuclear localization signal (Manchen et al., Biochem. Biophys. Res. Commun. (2001)287:1075-1082) and a caspase 3 cleavage site that triggers apoptosis when released from the BAT3 protein (Wu et al., J. Biol. Chem. (2004) 279-19264-19275).

### 2. Effects of BAT Sense and Antisense Constructs on ASFV DNA Production.

During the reconstitution experiments described above, we isolated a HeLatTA cell clone containing a construct (here designated as BAT3 1.3-3s) that contains the BAT3 EST in the sense direction relative to the Dox-controlled promoter (see below) and which showed a Dox-reversible decrease in ASFV titer that was quantitatively similar to the decrease observed for several cell clones expressing the BAT3 EST in the antisense direction (1.3-4as, 1.3-9as, and 1.2-14as) (Table 2, FIG. 8B).

The basis for defective ASFV virus production in HeLa cell clones when transfected with BAT3 1.3-3s or BAT3 1.3-4 as constructs was investigated in dot blot hybridization experiments using labeled ASFV DNA as a probe. In these experiments, the extent of viral DNA replication in different HeLa cells was inferred by quantitating viral DNA at different time points after ASFV infection. The hybridization signal on the dot blot shown in FIG. 4 indicates that the ASFV DNA in infected parental HeLa cells and in the BAT3 1.3-4as clone increased steadily from 16 h postinfection (hpi) to 48 hpi and leveled off at 72 hpi. In contrast, cells containing the BAT3 1.3-3s construct showed a decrease in the hybridization signal between 16 and 48 hpi, with a further decrease at 72 hpi.

These results argue that despite the similar ability of the BAT3 EST inserted into pLentiEST in the sense or antisense direction to limit ASFV virus production, the sense and antisense constructs had disparate effects on ASFV DNA replication.

### 3. Expression of ASFV Early and Late Genes in BAT3 EST Sense and Antisense Clones.

Production of the ASFV early and late proteins p30 and p72, respectively (Barderas et al., Arch. Virol (2001) 146:1681-1691; Borca et al., Virology (1994) 201-413-418; Gomez-Puertas et al., J. Virol. (1996) 70:5689-5694; Zsak et al., Virology (1993) 196: 596-602) was assayed by fluorescence microscopy following ASFV infection of HeLatTA cells containing the BAT3 1.3-3s or BAT3 1.3-4as constructs. At different times after infection, cells were fixed and the presence of these early or late proteins was determined using fluorescein-labeled antibodies generated against the ASFV p30 or p72 proteins. As seen in Table 3 (FIG. 8C), the fraction of cells showing staining for the p72 late protein was decreased dramatically at all time points in the BAT3 1.3-3s cells versus controls; the fraction of cells containing detectable p30 early protein was not affected 24 h after infection but, like the fraction containing p72, was sharply decreased at later times.

The BAT3 1.3-4as clone showed a very different pattern staining for ASFV early and late proteins: the cell fraction showing detectable p30 was unaffected by the BAT3 EST at any time, whereas cells that stained for p72 were increased by two- to three fold compared with the parental cell line. This result, together with the ASFV DNA dot blot data shown in FIG. 4, indicates that in the BAT3 sense clone, blockage of ASFV replication was affected by BAT3 1.3-3s at an early stage of the virus life cycle, whereas the effects of antisense-mediated interference with BAT3 expression did not occur until a much later stage.

### 4. Analysis of ESTs Present in the BAT3 1.3-3s and 1.3-4as Constructs and Their Effects on Cellular Gene Expression.

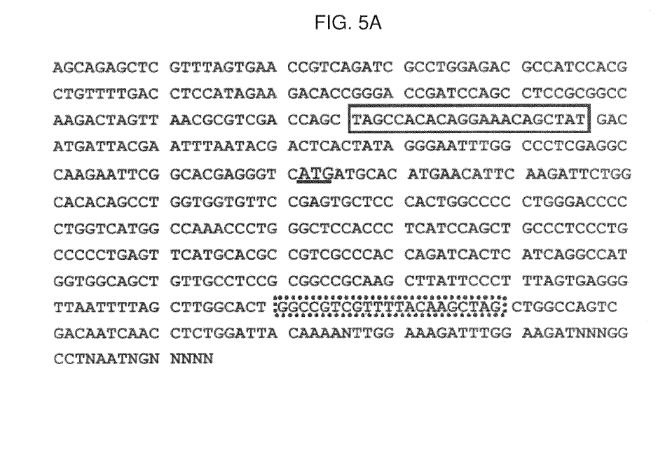

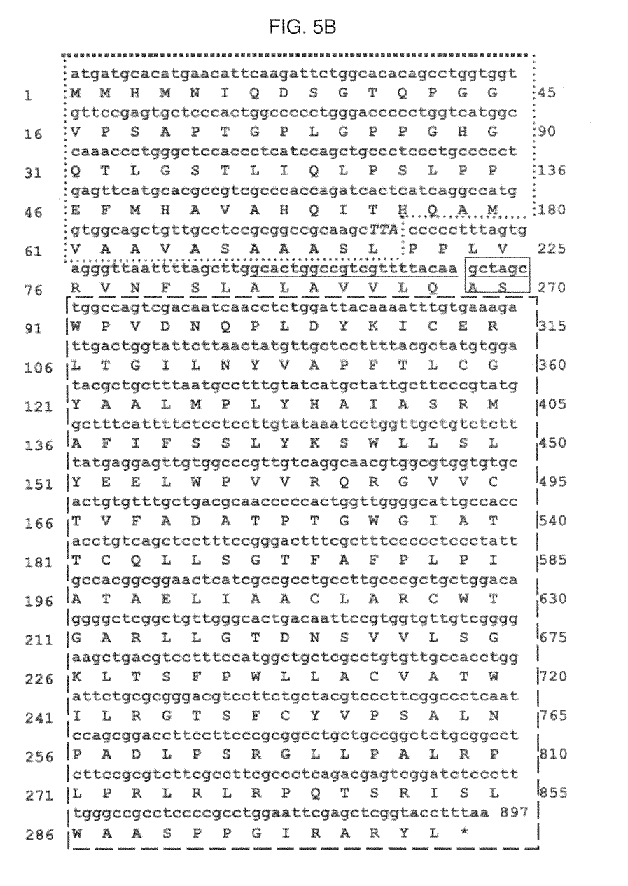

The BAT3 ESTs we cloned are contained within an NheI fragment introduced into the pLentivirus vector. The orientation of the BAT3 EST was determined by PCR amplification using a nested 3′ primer derived from the pLentiviral vector and one of the universal forward or reverse primers that flanks the EST fragment. Analysis of the sequence of the amplified PCR DNA product indicated that it encodes a predicted fusion protein of 299 amino acids consisting of an ORF starting in the BAT3 1.3-3s clone, at an ATG translation start codon (FIG. 5A) and extending into a vector-derived woodchuck hepatitis virus post transcriptional regulatory element (WTRE) (FIG. 5B) (Follenzi et al., Nat. Genet. (2000) 25:217-222).

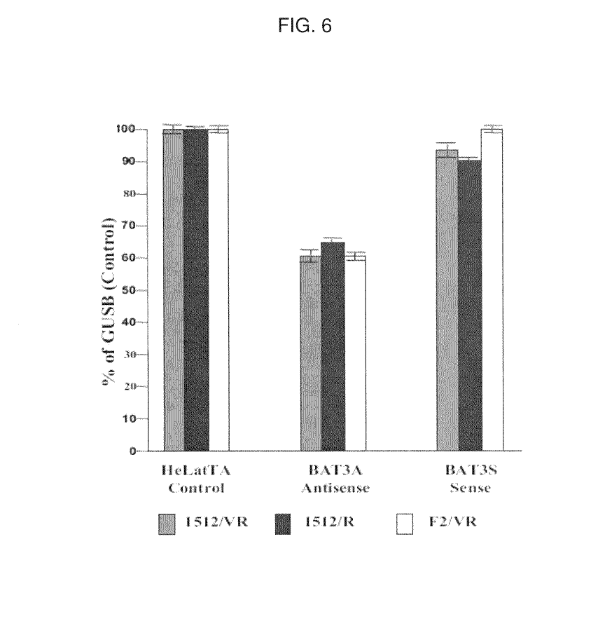

The BAT3-encoded segment of this predicted fusion peptide corresponds to amino acids 450 through 518 of the human native BAT3 as identified by accession number CAI18506. Three alternatively spliced transcript variants have been reported for human BAT3. Quantitative PCR analysis using oligonucleotide primers designed to detect cellular transcripts that include the segment of BAT3 present in the fusion protein showed an approximately 40% decrease in the abundance of these transcripts in three separate experiments (FIG. 6) in the antisense clone BAT3 1.3-1.3as. However, we were unable to detect a corresponding change in the overall abundance of BAT3 proteins by Western blotting using a polyclonal antibody generated against a synthetic peptide corresponding to the segment of BAT3 present as inserts in the BAT3 1.3 clones (data not shown), possibly because BAT3 protein epitopes in the peptide used to generate the antibody are predicted from sequence analysis to be present also in BAT3 protein isoforms that may not be affected by the transcript reduction we have observed. As expected, there was no detectable effect on the abundance of cellular BAT3 transcripts as a result of expression of the BAT3 insert in the sense direction, as shown in FIG. 6.

To learn whether the short vector-encoded peptide sequence fused to the C-terminal end of the BAT3-encoding EST that we cloned is required for antiviral effects observed for BAT3 1.3-3s, two additional constructs were tested for possible effects on ASFV production. In the first, the leucine codon TTA that specifies the last amino acid of the BAT3 peptide segment (FIG. 5B) was mutated to a TGA translation termination codon. In the second construct, the BAT3 EST was cloned into a plasmid vector that yielded a BAT3-encoded peptide lacking any vector-encoded component. Neither manipulation affected the ability of the BAT3 EST to reduce the ASFV titer, indicating that the observed effects on ASFV production are independent of the vector-encoded segment of the fusion peptide, as well as that of the lentivector-derived WTRE sequences.

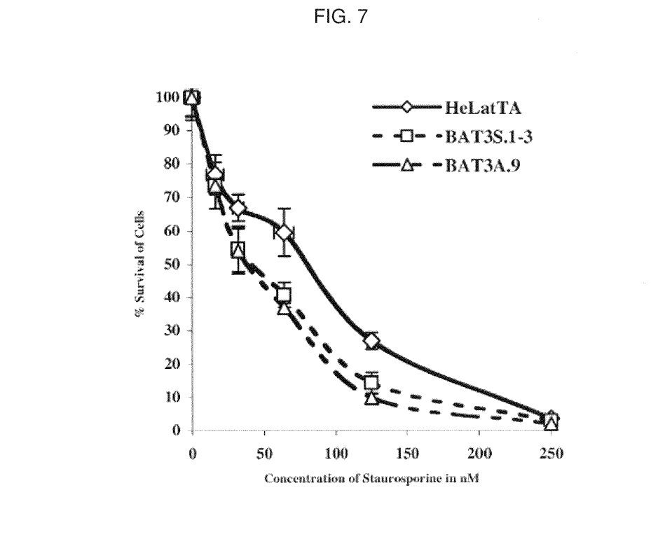

Previous work has shown that Scythe, a Drosophila melanogaster homolog of BAT3, can modulate apoptosis through its interaction with a protein called Reaper (Thress et al., EMBO J. (1998)17:6135-43; Thress et al., EMBO J. (2001) 20:1033-1041), and recent experiments have implicated BAT3 itself as a regulator of apoptosis in mammalian cells (Desmots et al., Mol. Cell. Biol. (2005)25:10329-10337). Microarray analysis of mRNA abundance in host cells carrying the BAT3 EST inserted in either the sense or antisense orientation showed that perturbation of host gene expression was observed in both types of cells. In cells containing the BAT3 EST expressed in the sense direction, 247 ESTs representing 63 previously annotated genes (functionally discriminating residue score of 0.049) were upregulated, and 133 ESTs representing 62 previously annotated genes were downregulated (functionally discriminating residue score of 0.091). Genetic ontology analysis indicated that half the number of genes whose expression was altered by the BAT3 1.3-3s construct, as shown in Table 4 (FIG. 8D), included those associated with cell cycle events, ubiquitination, or apoptotic processes, as well as those implicated in cell adhesion, signal transduction, and transcription, indicating that expression of the BAT3 1.3-3s EST has broad cellular effects. As shown in Table 4 (FIG. 8D), four proapoptotic genes were upregulated. In contrast with our results for BAT3 1.3-3s, the BAT3 antisense clone BAT3 1.3-4as showed upregulation for only 11 mRNA species, including two genes involved in cell cycle regulation: cyclin-dependent kinase 6 (Cdk6) and Mdm2 (s). The results from the same set of microarrays showed 81 genes that were downregulated; 37 of the 53 unique genes submitted to the Gene-Ontology software were annotated. Whereas uninfected cells expressing BAT3 1.3-3s and BAT3 1.3-3as grew normally in the absence of apoptotic agents, they showed increased sensitivity to the apoptosis-inducing agent staurosporine (FIG. 7), consistent with earlier evidence implicating BAT3 and its homologs in the modulation of apoptosis (Desmots et al., supra; Thress et al., (1998) supra; Thress et al., (2001) supra). Transcripts downregulated by the BAT3 1.3-3as clone included those encoded by genes related to proteins that have been localized to mitochondria, as shown in Table 5 (FIG. 8E). In this context, it is interesting that the Scythe protein of Xenopus laevis (Thress et al., (1998) supra) has been found to reversibly inhibit the actions of the heat shock chaperone protein Hsp70 (Thress et al., (2001) supra), which has a well-established key role in apoptosis (Nollen et al., Mol. Cell. Biol. (2000) 20:1083-1088; Takayama et al., EMBO J. (1997) 16:4887-4896).

### C. Discussion

In this report we have described the use of an EST-based genome-wide inactivation procedure to functionally identify host cell genes implicated in ASFV replication. Among the genes identified are loci involved in the host cell immune response, signal transduction, mitochondrial stability, and functions related to actin cytoskeleton reorganization. The role of these genes in ASFV production was confirmed by reconstitution of the defective production phenotype in naïve cells, by the independent discovery of CGEPs in separate screens (some involving different types of host cells), and by our ability to reverse genetic inhibition of ASFV production by downregulation of the tetracycline-controlled promoter used for expression of these ESTs.

We have focused here on the effects of an EST from a gene that encodes a segment of BAT3, a member of the BAG1 protein family (Bimston et al., EMBO J. (1998) 17:6871-6878; Takayama et al., J. Biol. Chem. (1999) 274: 781-786 found in the MHC III region of human chromosome 6 (Banerji et al., Proc. Natl. Acad. Sci. USA (1990) 87:2374-2378) and which has been reported to modulate apoptosis and cell proliferation during mammalian development (Desmots et al., Mol. Cell. Biol. (2005) 25:10329-10337). The observed effects of BAT3, which produces 25 different transcripts and putatively a large number of protein isoforms as a result of alternative splicing (National Center for Biotechnology Information (2005) [Online.] http://www(dot)ncbi(dot)nlm(dot)nih(dot)gov/IEB/Research/Acembly), on ASFV infection are of special interest.

Our data indicate that dysfunction of BAT3 in mammalian cells results in the upregulation of apoptotic genes and that such dysfunction in ASFV-infected cells is associated with impairment of ASFV replication. In uninfected cells, BAT3 dysfunction did not affect cell growth per se but was observed to enhance staurosporine-induced apoptosis. Taken together, our findings indicate that perturbation of apoptosis-related signaling may account for the observed effects of BAT3 dysfunction on ASFV replication.

Whereas we found that expression of the BAT3 EST in either the sense or antisense orientation resulted in a reduction of ASFV titer, the mechanism underlying this reduction appears to be substantively different for the sense and antisense constructs. Dot blot DNA hybridization results indicated that the replication of ASFV DNA was not affected by BAT3 antisense transcripts; however, an increase of the late viral protein p72 was observed, accompanied by a decrease in the production or release of infectious virus. In the BAT3 1.3-3s sense clone, virus functions were severely inhibited starting at an early stage of infection, as evidenced by delayed expression of the early viral protein p30 and reduced viral DNA replication. Moreover, the overall effects of the sense construct on the expression of host cell genes were more extensive than those of the antisense construct, suggesting more complete interference with BAT3 functioning, or possibly differential effects of the sense and antisense constructs on the actions of different BAT3 isoforms. Many of the transcripts affected by BAT3 1.3-3s were host response genes that react to external stimulation, while other transcripts affected by this construct are implicated in cell cycle regulation. Induction of the cyclin-dependent kinase inhibitor (p21), which also has been observed independently in cells infected with ASFV (Granja et al., J. Immunol. (2006) 176:451-462), was especially prominent. The ability of a short EST encoding 71 amino acids of BAT3 to extensively perturb cellular gene expression may occur by several mechanisms. These include possible dominant negative effects of competition between the peptide and native protein for a site on a BAT3 binding partner or reduced activity of native BAT3 by partial heterodimer formation with the peptide. While short peptide domains of larger proteins also can mimic the positive actions of a native protein (Agou et al., J. Biol. Chem. (2004) 279:54248-54257; Freed, Trends Microbiol. (2003) 11:56-59; Reeves et. al., Drugs (2005) 65:1747-1766), our finding that the BAT3 sense and antisense constructs both can interfere with ASFV production argues that the sense construct we have studied is affecting ASFV replication through a dominant negative mechanism.

Among the CGEPs identified by our screen was C1qTNFR6, which encodes a protein of the tumor necrosis factor superfamily (Shapiro et al., Curr. Biol. (1998) 8:335-338). We note that members of the C1q superfamily have been implicated in the recognition of viral and microbial surfaces and in TNF-mediated cell death (Shapiro et al., Curr. Biol. (1998) 8:335-338). Induction of the C1q complement component protein in conjunction with cytokines (e.g., TNF-α and interleukin-1α) has been observed in thymocytes of pigs infected with classical swine fever virus (Sanchez-Cordon et al., J. Comp. Pathol. (2002) 6127:239-248). C1q also has been shown to have a role in apoptosis of mononuclear blood cells, contributing to lymphopenia—one of the characteristic effects of ASFV infection (Sanchez-Cordon et al., Vet. Pathol. (2005) 42:477-488).

Another EST found to affect virus production when expressed in the antisense direction encodes a mitochondrial outer membrane translocase that is part of the TOM complex, which specifically mediates the transport of preproteins, e.g., apocytochrome c, a precursor of cytochrome c, and other precursor proteins into the mitochondrial intermembrane (Diekert et al., EMBO J. (2001) 20:5626-5635). Cytochrome c functions as an electron carrier between complex III and complex IV of the electron transport chain and is released into the cytoplasm in response to apoptotic stimuli. Potentially, reduction of TOM40 may limit the uptake of cytochrome c into the mitochondria and interfere with virus replication by affecting the electron transport chain and, consequently, apoptosis.

The gene silencing procedure described here has proven useful in identifying genes and proteins whose perturbed function limits ASFV replication. We suggest that such CGEPs may be targets for pharmacological or immunological interventions that treat ASF. Agents that mimic the effects of the BAT3 dominant negative peptide may prove to be of particular value in this regard.

### II. Identification of Genes Associated With FMDV Susceptibility

### A. Materials and Methods

### 1. Cell Culture and Virus

Bovine kidney (LF-BK) cells and resistant derivatives cells were maintained in Dulbecco's modified Eagle medium (DMEM, Gibco) supplemented with 10% FBS. LF-BK cells were originally obtained from the Foreign Animal Disease Diagnostic Laboratory (FADDL) at the Plum Island Animal Disease Center (Swaney L., Vet. Microb. (1987)18:1-14; Baxt B., Virus Res. (1987) 7: 257-271). FMDV strain O/UK/2001, porcine enterovirus, swine vesicular disease virus (SVDV), encephalomyocarditis virus (EMCV) and vesicular stomatitis virus (VSV) were provided by Fred Brown. Procedures for infections and plaque assay with FMDV were described previously (Chinsangaram et al. J. Virol. (1999)73: 9891-9898). The number of cells and multiplicity of infection (MOI) were calculated in each experiment.

### 2. Generation of LF-BK EST Cell Library

The target cell lines: Hela, a cell line derived from cervical cancer and a human metastatic melanoma cell line HT144 (Gouon et al., Int. J. Cancer (1996) 68: 650-662; Fogh et al., J. Natl. Cancer Inst. (1997) 59:221-226), were made to overexpress tTA (tetracycline-dependent transcriptional activator) by infection with a pBabetTAPuro retrovirus. We then generated plentiEST libraries in both HelatTA and HT144tTA cell lines as described Lu et el., supra. Briefly, 20 subconfluent 15 cm² plates of HelatTA and HT144tTA1 cells (ca. 2×10⁷ cells in total) were infected with 500 μl of pLentiEST virus supernatant derived from 293T packaging cells and pseudotyped with the VSV-G envelope gene in the presence of 4 μg/ml of polybrene. Following G418 selection (1,000 μg/ml for HeLatTA and 600 μg/ml for HT144 tTa), the G418 resistant clones were pooled in several aliquots and expanded one generation to form the plentiEST library of HelatTA and HT144tTa respectively.

The generation of the plentiEST library in LF-Bk cell line was performed as previously described in Lu et al., sukpra). Briefly, A LF-BK tTa cell line was made by the infection of a pBabetTaPuro retrovirus carrying the tetracycline-dependent transcriptional activator (tTA) under the control of the cytomegalovirus (CMV) promoter in the presence of 5 μg/ml polybrene (Sigma). The infected cells were then subjected to 2 μg/ml puromycin selection for 2-3 weeks. The tTA activity of puromycin resistant cell clones were tested by using a luciferase reporter gene under the control of a Tet Response Element (TRE) promoter (Gossen, et al., supra). A cell clone that showed high luciferase activity and good regulation of this reporteer gene by Tc (53 fold) was selected and infected with the plentiEST library which contains a collection of ˜40,000 human expressed sequence tags (EST) under the control of TRE-CMV. LF-BK EST cells were selected by using 1 mg/ml of G418 (Gibco) for 34 weeks.

### 3. FMDV Growth Assay

Cell monolayers were infected with FMDV O/UK/2001 at MOI 10 or 0.1. After 1 h, the cells were washed with 150 mM NaCl, 20 mM MES buffer pH 6.0 to inactivate unabsorbed virus and then incubated in DMEM at 37° C. Supernatant samples were collected at intervals and titrated on BHK-21 cells. TCID₅₀ was determined as described by Reed and Muench, Am. J. Hyg. (1938) 27: 493-497.

### 4. Infectious-Center Assay

tTA LF-BK and resistant cells were infected with FMDV O/UK/2001 at an MOI of 10. After 1 h of adsorption, the cells were trypsinized, washed once with DMEM, once with 150 mM NaCl, 20 mM MES buffer pH 6.0 to inactivate residual virus and once more with growth medium. These cells were diluted 10-fold, mixed with 6×10⁵ LF-BK cells per sample and seeded into six-well plates. At 48 hours post infection (hpi), plates were fixed and stained. The percentage of infected cells was determined from the number of plaques obtained in the plaque assay relative to the number of tTA LF-BK or Entpd6 cells originally infected.

5. RNA Extraction and PCR Amplification of vRNA

Cytoplasmic RNA was extracted from cells according to the RNeasy protocol (Qiagen). Reverse transcriptions were done with Super Script RT (Invitrogen) using random hexamers. PCR amplification of FMDV RNA was carried out using the antisense 5′TCAGGGTTGCAACCGACCGC3′ (SEQ ID NO:05) and sense 5′TTCGAGAACGGCACG GTCGG3′ (SEQ ID NO:06) primers corresponding to the 3D genomic region (König and Piccone, unpublished).

### 6. DNA Extraction and PCR Amplification of EST Insert

Genomic DNA was isolated from cells by the Red Extract-N-Amp Tissue PCR (Sigma) kit following the manufacturers' instructions. Primers to amplify the EST insert were specific for the retroviral vector 5′CATAGCGTAAAAGGAGCAACA3′ (SEQ ID NO:07) and 5′TCTGCTAGCCACACAGGAAACAGCTATG3′ (SEQ ID NO:08) or 5′TCTGCTAGCTTGTAAAACGACGGCCAGTG 3′ (SEQ ID NO:09) depending on their insertion into the vector. As a consequence, the EST-RNA transcripts were expressed in the sense or antisense orientation to the CMV promoter. The EST orientation was determined by DNA sequencing and their identity determined by genomic library screening.

### 7. Northern Blot Analysis

tTA LF-BK and resistant cell clones were infected at MOI 10 or MOI 0.1 with FMDV O/UK/2001 for 1 h at 37° C. Cytoplasmic RNA was extracted at various times post infection according to the RNeasy protocol (Qiagen). Equal amounts of total RNA were separated on a 1% denaturing formaldehyde gel, blotted to a nylon membrane and hybridized with a ³²P-labeled antisense RNA corresponding to the 3D region of the FMDV genome. The same blots were stripped and reprobed for the βactine gene as a loading control.

### 8. Western Blot Analysis and Immunofluorescence Microscopy

Cells were infected at an MOI of 10 and at 4 hpi mock and infected cells lysates were fractionated on a 4-12% SDS-PAGE and blotted onto PDVF membranes. Western blot analyses were performed using antiserum prepared against 3D polymerase (kindly provided by H. Wang, FADDL) and visualized by chemiluminescence (InvitroGen). An anti-tubulin Mab was purchased from Lab Vision. Immunofluorescence was performed as described by O'Donnell et al. Virology (2001) 287: 151-162. Briefly, tTA or Endtpd 6 cell clones grown on coverslips (EMS) were infected as described above. At 4 hpi cells were fixed, permeabilized and immunostained using a pool of Mabs 10GA4.2.2 and 12FE9.2.1 (kindly provided by M. Grubman) against structural proteins of FMDV type O (Stave et al. J. Gen. Virol. (1988)67: 2083-2092). A Fluor-labeled goat anti-mouse IgG was used as secondary antibody (Molecular Probes). Cells were imaged in a Leica TCS SP2 confocal microscope.

### B. Results

### 1. Construction of an EST Library in LF-BK Cell Line and Isolation of FMDV Resistant Cell Clones

A variety of cell lines previously have been shown to be susceptible to FMDV infection, including BHK-21 (baby hamster kidney), LF-BK (bovine kidney) (Stave, supra; Baxt, supra) and IB-RS-2 (pig kidney). However, as our experimental protocol involved a screen for cell clones that survive FMDV infections, and spontaneous survivors represent an undesirable background in such phenotype-based screens, we carried out an initial analysis of the relative efficiency of infection in different cell lines to identify one that consistently showed a survival frequency of less than 10⁻⁶ following infection by FMDV under the conditions we employed. The LF-BK cell line was chosen for EST library construction because of its high susceptibility to FMDV infection and the absence of surviving cells after 4 sequential rounds of infection.

As described above, G418 resistant LF-BK tTA cells infected with a pLEST EST library were isolated and expanded by culture in DMEM for FMDV screening. Both naïve LF-BK tTA cells and LF-BK tTA EST library cells were seeded at 2×10⁶ cells per T25 flask and infected 2 h later with FMDV O/UK/2001 at an MOI of 10. After 2 days, cells were placed in DMEM and incubated at 37° C. for several days. The cultures were refed every 2 days and monitored for both viral-induced cytopathology and for outgrowth of virus resistant cell clones. Surviving cells were recovered and challenged with FMDV again as described above. After a total of four rounds of FMDV infection, the surviving cells were cloned and expanded into a monolayer. No surviving clones were obtained after four rounds of culture of parental LF-BK cells uninfected with the pLEST library.

About 180 surviving cell clones were isolated and each was tested for the ability to produce FMDV in a virus plaque assay. This showed a range of limitation of virus production among the different clones. PRC-amplified genomic DNA dontaining ESTs present in surviving clones that showed a reduction in FMDV titer by at least 99% was sequenced (as reported in Lu et al., supra and described in Materials and Methods). Annotated information for the genes represented by each of the sequences obtained is shown in Table 6 (FIG. 15). Certain of the ESTs by this procedure identified correspond to genes that previously have been implicated in viral infections, including IRF7 (interferon regulatory factor 7) and SPP (signal peptide peptidase).

Among the surviving clones, one that contains the EST insert of NTPDase 6 showed especially prominent reduction of FMDV production. NTPDase 6 belongs to a family of enzymes which are known to modulate a variety of important cellular responses by controlling extracellular nucleotide concentrations. Unlike other members in the NTPDase family, NTPDase 6 exists as both a soluble and membrane bound form. Although NTPDases have not previously been shown to be related to viral infection, the strong FMDV resistance phenotype associated antisense of an NTPDase 6 EST suggested this enzyme may be necessary for FMDV replication and prompted us to further investigate the properties of the LF-BHK cell clone containing the NTPDase 6 EST.

### 2. Reconstitution of the FMDV Resistant Phenotype With the Expression of Antisense NTPDase 6 EST

In order to confirm that the FMDV resistant phenotype exhibited in the NTPDase 6 clone was a result of the expression of antisense EST vector rather than that of spontaneous mutations in the cell clone or the insertion position of EST, we introduced the vector containing the NTPDase 6 EST into naïve LF-BK cells and tested for reconstitution of the FMDV resistance phenotype.

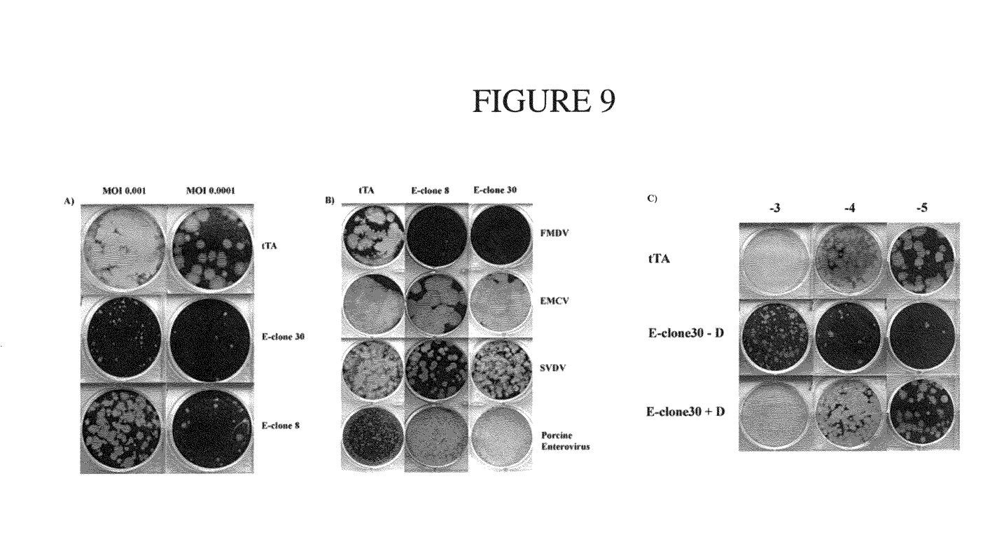

Approximately 30 reconstituted LF-BK tTA cell clones expressing the antisense RNA of NTPDase 6 est were amplified and retested for limitation of production of FMDV O/UK/2001. These clones exhibited variation in their ability to limit virus production, possibly due to different levels of expression of NTPDase 6 antisense RNA expected from integration of the vector/EST at different chromosomal sites in different clonal isolates. Especially prominent limitation was observed for FMDV O/UK/2001 reconstitution clones 8 and 30 (FIG. 1, panel A), and these were chosen for additional studies. When compared to LB-BK tTA cell line in plaque assay using FMDV O/UK/2001, the clones that showed most prominent limitation in virus production, clones 8 and 30, significantly reduced the number and size of plaques (FIG. 9 panel A). A similar result was obtained with FMDV isolates from serotypes A (A24) and C (C₃ Resende). In addition, challenge of the reconstituted clones with other picornoviruses, including porcine enterovirus, SVDV, or EMCV, showed that the clones transcribing the NTPdase 6 in the antisense direction are as susceptible to these viruses as the naïve cell lines (FIG. 9, panel B), suggesting the FMDV resistance of the reconstituted clones 8 and 30 is specific to FMDV.

Clones 8 and 30 were examined for dependence of the FMDV resistance phenotype on transcription from the Tc-regulated promoter in the pLenti vector by adding doxycyclin to turn off transcription into the EST. Plaque assays showed that both clones acquired the ability to produce control levels of FMDV with the addition of doxycyclin (FIG. 9, panel C), confirming that the virus resistant phenotype is dependent on the expression of the inserted EST.

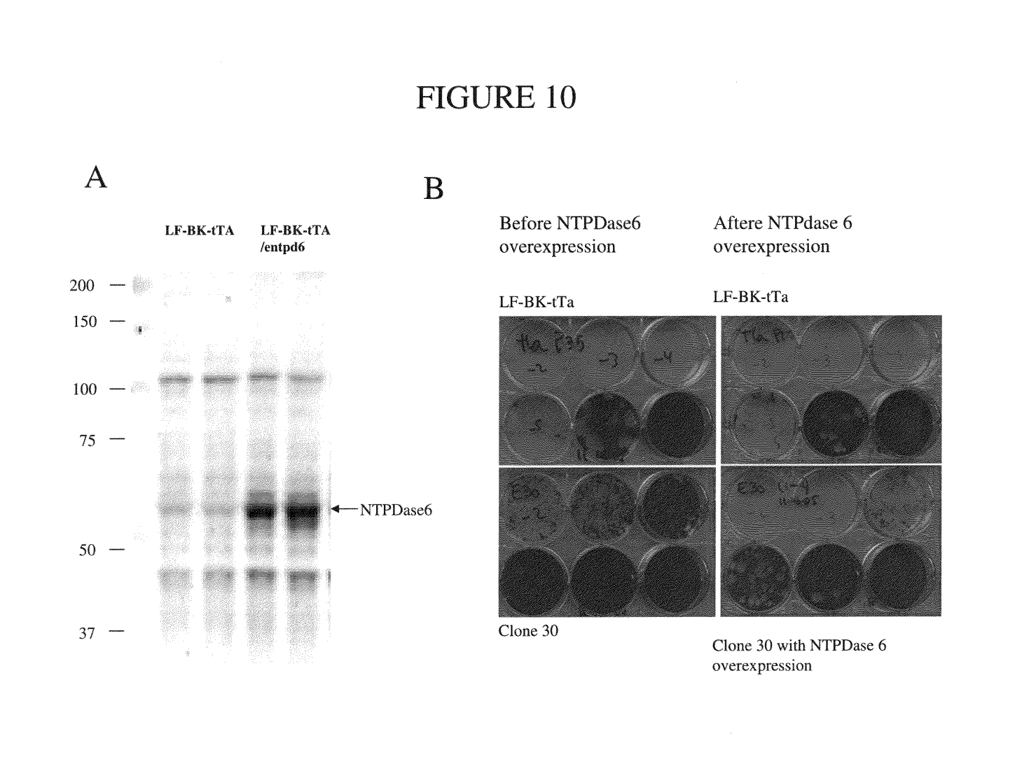

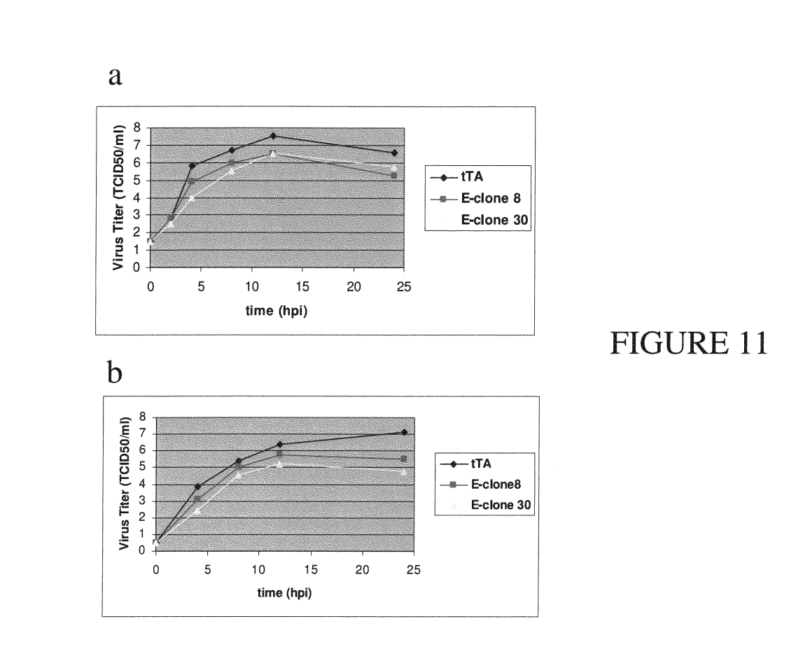

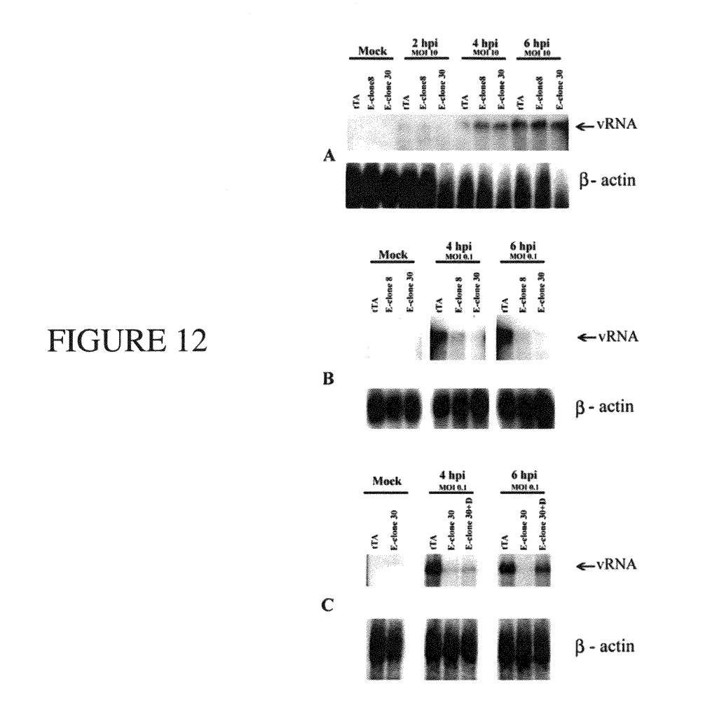

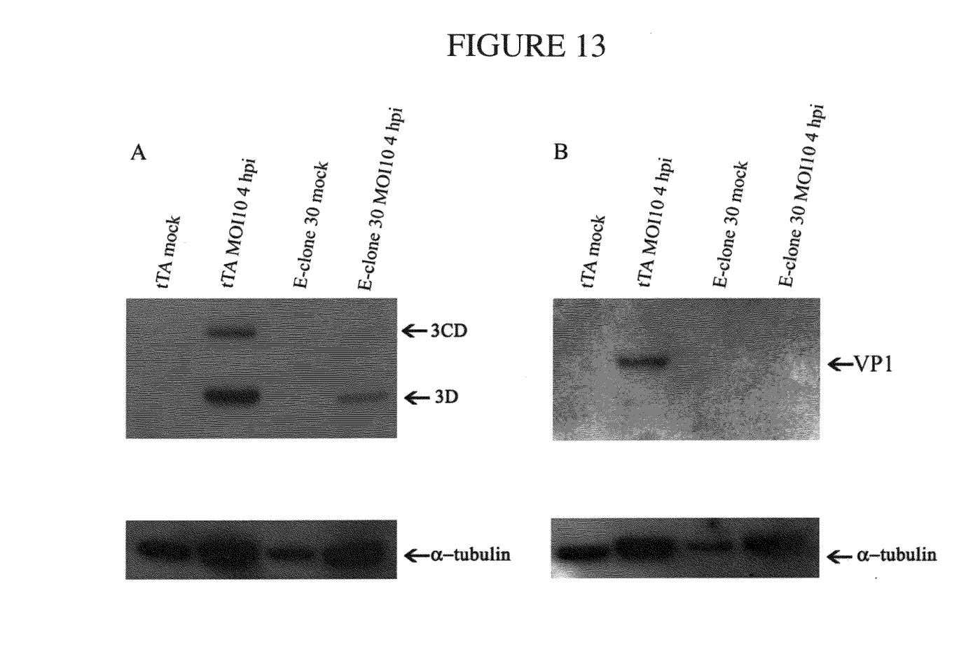

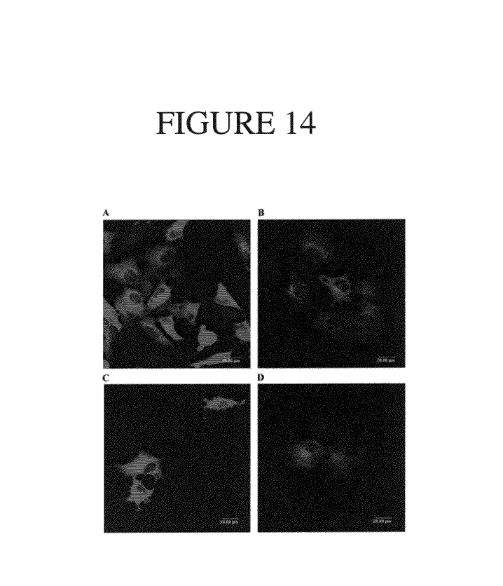

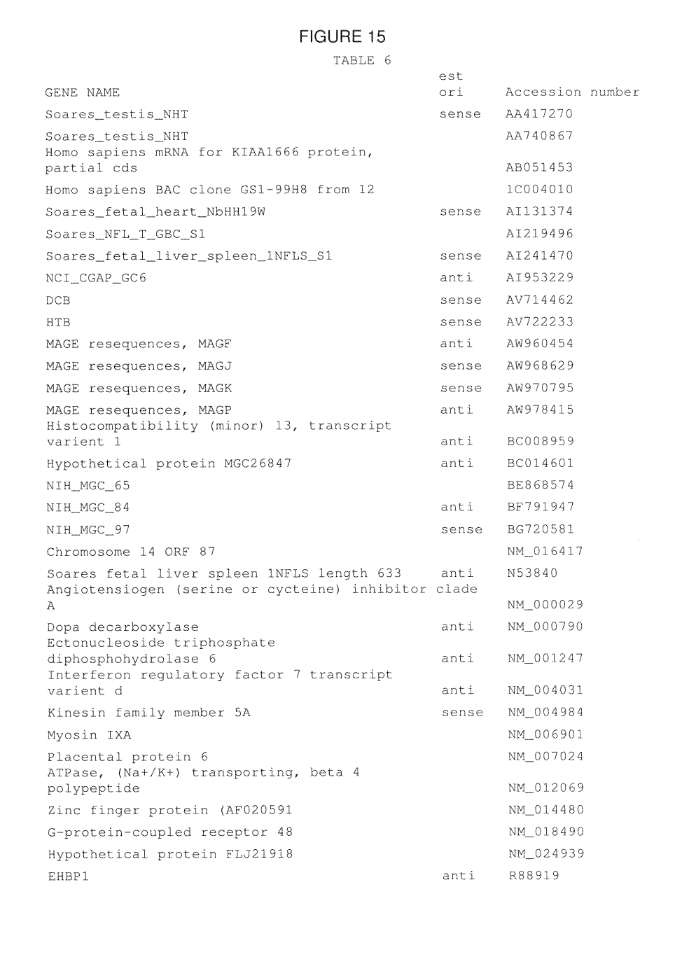

We also put a NTPdase 6 overexpression construct into the wild type LF-Bk/tTa cell line and found that the overexpression of NTPDase 6 did not make the cells more sensitive to FMDV infection (data not shown). However, when we put the same construct into clone 30, the overexpression of NTPDase6 can be detected by Western blotting analysis and the reverse of the virally resistant phenotype was observed (FIG. 10), indicating the viral production can be brought back to the normal level by elevating NTPDase 6 expression level.

### 3. Characterization of NTPDase 6 Mediated-Resistant Cells

Having demonstrated that expression of antisense NTPDase 6 can limit the ability of LF-BK cells to produce FMDV, we further analyzed how the virus grows in the reconstituted clones compared to wild type cell line. Cells were infected with FMDV O/UK/2001 at high and low multiplicity of infection (MOI 10 or 0.1) and samples titrated in BHK-21 cells (FIG. 11, panels a and b respectively). At MOI 10, one-step growth curves showed that virus replicated more slowly in naïve cells engineered to produce antisense RNA to NTPDase 6 cell clones and resulted in 10 fold less virus compared to LF-BK tTA cells. The viral growth difference became even more significant at MOI 0.1 with about 50-200 folds less viral production in the NTPDase 6 cells.

We then investigated the stage of the viral life cycle that was blocked in the antisense-NPDase6, evaluating viral replication by Northern blot analysis. As shown in FIG. 12A comparable levels of viral RNA were detected in a time course at MOI 10. However at MOI 0.1 (FIG. 12B) viral RNA accumulation was markedly reduced in both reconstituted clones (i.e. 8 and 30) compared to the parental LF-BK tTa cells. No viral RNA degradation was observed. In addition, when the cells were cultivated in the presence of doxycyclin, an increase of viral RNA production was observed in clone 30 (FIG. 12C), indicating the vRNA synthesis depending on the expression of antisense NTPDase 6 EST.

The expression of viral proteins in NTPDase 6 and LF-BK tTa cells was examined by Western blot analysis and immunofluorescence microscopy. The production of the viral polymerase (3D) (FIG. 13A) and the viral structural proteins (FIG. 13B) were significantly reduced in clone 30 compared to LF-BK tTA cells, assessed by Western blotting. For immunofluorescence microscopy, clone 30 and LF-BK tTA cells were infected with FMDV at an MOI of 10 and stained with structural proteins-specific Mabs 10GA4.2.2 and 12FE9.2.1. At 2 hpi, both cells had staining that was homogeneously distributed in the cytoplasm and showed similar fluorescence. At 4 hpi, although an equivalent percent (higher that 95%) of cells expressing viral proteins were detected, the immunofluorcence intensity was much lower in the cells of clone 30 (FIGS. 14A and B). Quantitation of fluorescence intensity determined for individual infected cells showed an 85% reduction in the cells expressing antisense to NPTDase 6.

The above results of immunofluorescent microscopy indicate that entry of FMDV viruses is not defective in cells expressing antisense to NTPD6, as the number of infected cells and the concentration of viral proteins are about the same for both reconstituted and naïve cells at early time points. We carried out infectious center assays in clone 30 to test directly whether this clone can be efficiently infected by FMDV. Both clone 30 and LF-BK tTA cells were infected with O/UK/2001 at MOI 10 and treated with low pH saline buffer to inactivate residual virus. Cells were trypsinized and seeded with cells sensitive (tTA LF-BK) or resistant (NTPDase 6) to FMDV. After 4 h incubation to allow cell attachment, the cultures were incubated under gum tragacanth for 48 h. The percentage of infected cells was approximately 64% for tTA LF-BK and 80-55% for NTPDase 6 cells, indicating a comparable virus binding efficiency in both cells. We also observed that infected NTPDase 6 cells seeded in a sensitive cell background developed the same plaque size as infected tTA LF-BK, whereas infected tTA LF-BK or NTPDase 6 cells developed a very small plaque size in a Entpd6 cell background.

The above results show that application of the EST-based gene-inactivation approach in accordance with aspects of the invention allowed the development of FMDV resistant cells mediated by inhibition of specific cellular genes. A cell library, susceptible to the FMDV infection, containing altered expression of a single cellular gene was screened for virus surviving and resistant cell clones were isolated. An FMDV resistant cell line produced by antisense expression of the ectonucleoside triphosphate diphosphohydrolase 6 (Entpd6) gene was derived from the virus-sensitive LF-BK cell line (hereinafter referred to as Entpd6 cell clones). Two cell clones, 8 and 30, were characterized in detail and different steps of viral cycle in these cells were compared to the parental tTA LF-BK cells. Cellular resistance was observed to be specific for FMDV. The viral RNA and protein synthesis were significantly reduced in Entpd6 cell clones. The data show that replication and/or assembly in these resistant cells is inefficient or defective. The data indicate that the resistant cells might be deficient in some cellular factor(s) required for efficient replication.

Ecto-nucleoside-triphosphate diphosphohydrolase-6 (Entpd6), also known as CD39L2, belongs to a family of ecto-nucleoside triphosphate diphosphohydrolases (E-NTPDase family). The enzyme occurs in a membrane-bound form and in a soluble extracellular form. It has been associated with the Golgi apparatus and to a small extent also with the plasma membrane (Braun et al. Biochem. J. (2000) 351:639-647).

The results reported here demonstrate that cellular genes from susceptible cells can be modified to inhibit virus multiplication. Their identification and subsequent engineering in transgenic animals will provide constitutive resistance to FMDV and serve as a new approach to disease control.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method of identifying a mammalian cellular gene exploited by a viral pathogen (CGEP), said method comprising:
(a) transforming a population of mammalian cells with a random homozygous knockout (RHKO) library to produce an RHKO cellular library;
(b) challenging said RHKO library with a virus;
(c) identifying a member(s) of said RHKO cellular library that is resistant to infection by said virus; and
(d) determining which gene in said identified member(s) of said RHKO cellular library has been inactivated by a member of said RHKO library to identify a mammalian CGEP.

2. The method according to claim 1, wherein said RHKO library is an RHKO/GSV library.

3. The method according to claim 1, wherein said RHKO library is an RHKO/EST library.

4. The method according to claim 1, wherein said virus is a double-stranded DNA virus.

5. The method according to claim 4, wherein said double-stranded DNA virus is an Asfarviridae.

6. The method according to claim 5, wherein said Asfarviridae is an African Swine Fever Virus.

7. The method according to claim 1, wherein said virus is a single-stranded RNA virus.

8. The method according to claim 7, wherein said single-stranded RNA virus is a Picornaviridae.

9. The method according to claim 8, wherein said Picornaviridae is a Foot-and-Mouth Disease Virus.

10. A method of treating a subject suffering from a virally mediated disease condition, said method comprising:
administering to said subject an effective amount of CGEP inhibitory agent to treat said subject.

11. The method according to claim 10, wherein said virally mediated disease condition is an Asfarviridae disease condition.

12. The method according to claim 11, wherein said Asfarviridae disease condition is an African Swine Fever Virus disease condition.

13. The method according to claim 12, wherein said CGEP is chosen from BAT3, C1qTNF and TOM40.

14. The method according to claim 13, wherein said CGEP is BAT3.

15. The method according to claim 10, wherein said virally mediated disease condition is a Picornaviridae disease condition.

16. The method according to claim 15, wherein said Picornaviridae disease condition is a Foot-and-Mouth Disease Virus disease condition.

17. The method according to claim 16, wherein said CGEP is NTPDase 6.

18. The method according to claim 10, wherein said subject is an unregulate.

19. The method according to claim 10, wherein said subject is a human.

20. A method of conferring a virally resistant phenotype on a subject, said method comprising:
administering to said subject an effective amount of a CGEP inhibitory agent.

21. The method according to claim 20, wherein said virally mediated disease condition is an Asfarviridae disease condition.

22. The method according to claim 21, wherein said Asfarviridae disease condition is an African Swine Fever Virus disease condition.

23. The method according to claim 22, wherein said CGEP is chosen from BAT3, C1qTNF and TOM40.

24. The method according to claim 23, wherein said CGEP is BAT3.

25. The method according to claim 20, wherein said virally mediated disease condition is a Picornaviridae disease condition.

26. The method according to claim 25, wherein said Picornaviridae disease condition is a Foot-and-Mouth Disease Virus disease condition.

27. The method according to claim 26, wherein said CGEP is NTPDase 6.

28. The method according to claim 20, wherein said subject is an unregulate.

29. The method according to claim 20, wherein said subject is a human.

30. A transgenic non-human mammal having a viral infection resistant phenotype that is conferred upon said mammal by a modification in a CGEP.

31. The transgenic mammal of claim 30, wherein said mammal is an unregulate.

32. The transgenic mammal of claim 31, wherein said mammal is resistant to African Swine Fever Virus infection.

33. The transgenic mammal of claim 32, wherein said CGEP is chosen from BAT3, C1qTNF and TOM40.

34. The transgenic mammal of claim 31, wherein said mammal is resistant to Foot-and-Mouth Disease Virus infection.

35. The transgenic mammal of claim 34, wherein said CGEP is NTPDase 6.

36. A system for identifying a mammalian cellular gene exploited by a viral pathogen (CGEP), said system comprising:
(a) a random homozygous knockout (RHKO) library;
(b) mammalian cells; and
(c) a virus.


SEQUENCE LISTING (9 sequences)

SEQ ID NO: 1 - DNA, 23 bp, human
gtggaacccgtggtcatgatgca

SEQ ID NO: 2 - DNA, 22 bp, human
gtcatgatgcacatgaacattc

SEQ ID NO: 3 - DNA, 19 bp, human
ggtggagcccagggtttgg

SEQ ID NO: 4 - DNA, 20 bp, human
cctgctgtcccagggtttgg

SEQ ID NO: 5 - DNA, 20 bp, foot and mouth disease virus
tcagggttgcaaccgaccgc

SEQ ID NO: 6 - DNA, 20 bp, foot and mouth disease virus
ttcgagaacggcacggtcgg

SEQ ID NO: 7 - DNA, 21 bp, Foot and mouth disease virus
catagcgtaaaaggagcaaca

SEQ ID NO: 8 - DNA, 28 bp, foot and mouth disease virus
tctgctagccacacaggaaacagctatg

SEQ ID NO: 9 - DNA, 29 bp, foot and mouth disease virus
tctgctagcttgtaaaacgacggccagtg